SEQUENCE LISTING (35 sequences)

SEQ ID NO: 1 - protein, 5 aa, synthetic construct
TYAIS

SEQ ID NO: 2 - protein, 17 aa, synthetic construct
GIIPIFGKAHYAQKFQG

SEQ ID NO: 3 - protein, 14 aa, synthetic construct
KFHFVSGSPFGMDV

SEQ ID NO: 4 - protein, 11 aa, synthetic construct
RASQSVSSYLA

SEQ ID NO: 5 - protein, 7 aa, synthetic construct
DASNRAT

SEQ ID NO: 6 - protein, 8 aa, synthetic construct
QQRSNWPT

SEQ ID NO: 7 - protein, 5 aa, synthetic construct
NYYIF

SEQ ID NO: 8 - protein, 17 aa, synthetic construct
IINPVDGDTNYNPSFQG

SEQ ID NO: 9 - protein, 7 aa, synthetic construct
GGYTMDR

SEQ ID NO: 10 - protein, 16 aa, synthetic construct
RSSQSLLHSNGYNYLH

SEQ ID NO: 11 - protein, 7 aa, synthetic construct
KVSNRFS

SEQ ID NO: 12 - protein, 9 aa, synthetic construct
FQNTHTPRT

SEQ ID NO: 13 - protein, 11 aa, synthetic construct
GPAPELLGGGS

SEQ ID NO: 14 - protein, 11 aa, synthetic construct
GPAPLGLGGGS

SEQ ID NO: 15 - protein, 5 aa, synthetic construct
GGGGS

SEQ ID NO: 16 - protein, 10 aa, synthetic construct
GGGGSGGGGS

SEQ ID NO: 17 - protein, 15 aa, synthetic construct
GGGGSGGGGSGGGGS

SEQ ID NO: 18 - protein, 7 aa, synthetic construct
GPAPELL

SEQ ID NO: 19 - protein, 13 aa, synthetic construct
PPCPAPELLGGGS

SEQ ID NO: 20 - protein, 13 aa, synthetic construct
PPCPAPLGLGGGS

SEQ ID NO: 21 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 22 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 23 - protein, 116 aa, synthetic construct
QVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 24 - protein, 112 aa, synthetic construct
DIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 25 - protein, 683 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 443 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 2109 bp, synthetic construct
  source 1..2109: mol_type = other DNA
atgggatggaccctcgtgttcctctttctgttgtccgtgaccgccggagtccacagccaagtccaactggtgcagtccggtgccgaagtgaaaaagcccggatcctcggtgaaagtgtcgtgcaagacctcgggagacactttctcgacctacgccattagctgggtcagacaggccccgggccagggcctggaatggatgggtggaattatccccattttcggaaaggcccactacgcgcagaaattccagggccgggtcaccattactgccgatgagagcacctcgactgcctacatggaactgtcgtcgctgaggtccgaggataccgccgtgtacttctgcgcccggaaattccatttcgtgagcggttcgccgttcggaatggatgtctggggacagggcactaccgtgaccgtgagcagcgcctccacaaaggggccatccgtattcccacttgctccttctagcaagtccacatcaggtgggacagcagctctgggctgtctcgtgaaggactactttcctgaacctgttactgtgtcctggaatagcggcgccttgacctctggcgtacatacctttccagcagtgctccagtcctcagggctgtattctctgtctagtgttgtaaccgtgcccagttctagcttgggtactcagacctacatctgcaatgtgaaccacaagccctcaaacacaaaagtcgacaagaaggtggagcctaaatcctgcgggccagcccccgagctgcttggaggagggtcccaagtgcagctcgtgcagagcggggctgaggtcaagaagcctggagagtccctgaagatctcctgcaagggaagcggatacaccttcaccaactactatatcttttgggtgcgccagatgccgggaaagggactcgagtggatgggcatcattaacccggtggacggcgacactaactacaacccgagcttccaaggacaagtgaccatctcagcggacaagtccatttccactgcgtaccttcaatggtcatcgcttaaggcgtcggacacggctatgtactactgtgcaagaggaggatacaccatggatcgctggggccagggcactcttgtgaccgtgtcatcagcgtccaccaagggtccctccgtgttccctctcgcgccgtcctcaaagtctacctccggtggaactgccgcgctcggttgtctcgtgaaggactacttcccggagcctgtgactgtctcctggaactccggggccctcaccagcggagtgcacactttccccgccgtgctgcaatcctccggcctgtacagcctgtcctccgtcgtgactgtgcctagctcctccctgggaacccagacctacatctgcaacgtgaaccacaagccctccaacaccaaggtcgacaagaaggtcgaaccgaagtcgtgcgacaagactcatacgtgccctccttgcccggccccggaactgctgggaggcccatccgtgttcctgttcccacccaagcctaaggataccctgatgatcagcagaacaccggaagtgacctgtgtggtggtggacgtcagccacgaagatcccgaggtcaagttcaattggtacgtggacggggtggaggtgcacaacgcaaagaccaagccccgggaggaacagtacaactccacctatcgcgtggtgtcggtgctgacggtgctgcaccaggactggttgaacggaaaggagtataagtgcaaagtgtcgaacaaggccctgcccgctcctatcgaaaagaccatctccaaggccaagggccagccgcgggaaccccaggtctacactctcccaccgagccgcgacgaactgactaagaatcaagtgtcgctgacttgcctcgtcaagggcttctacccgtccgacatcgccgtggaatgggagagcaacggccagccggaaaacaactacaagaccacccctcccgtgctggattccgacgggtccttcttcctgtactcaaaactgaccgtggataagtccagatggcagcagggcaatgtcttttcatgctccgtgatgcacgaggctctgcataaccactacacccagaagtcgctgtccctgtccccggggtaatga

SEQ ID NO: 28 - DNA, 1395 bp, synthetic construct
  source 1..1395: mol_type = other DNA
atggtgtcctccgctcaattcctcggacttcttctcctgtgtttccaaggcacccgctgtgaaatcgtgctgactcagtcacccgccactctgtcactttcacccggggagagggccactctgagctgccgggccagccagagcgtgtccagttacctggcctggtaccagcaaaagcccggacaggccccgagactgctgatctacgatgcatccaaccgcgccactggtattcctgcccggtttagcggctccggttccgggaccgacttcactctcaccatttcctccctggaaccggaagatttcgccgtgtactactgccagcagcgatccaactggccaacgtttggacagggaaccaaggtagagatcaaacggaccgtcgctgccccttcggtattcatctttccgccttcggacgaacagttgaaaagcggaacagcctcggtcgtgtgcttgctcaacaacttctatcccagagaggcgaaagtccagtggaaggtagacaacgctctccagtcaggaaactcgcaagagagcgtaaccgaacaggattcgaaggactccacctactccctgtcgagcacgctgacactctcaaaagcggattatgagaaacataaagtatatgcatgcgaggtcacgcaccagggtctgtcctcgcccgtgacgaaaagcttcaatcgcggcgagtgcggaccggccccgctgggcctgggcggtggatccgacattgtgatgacccaatctcccctgagcttacctgtgacccctggggaaccagcgagcattagctgcagatccagccagtccctgctgcactcaaacggatacaattatctgcattggtacctccagaagcccggccagtccccccaactcctgatctacaaggtgtccaaccggttctccggagtgccggaccggttctcgggctcgggctccggcactgacttcaccttgaagatctcgcgggtggaagctgaagatgtcggcgtttactactgtttccaaaacacccacaccccacgcactttcggaggcggaactaaggtcgagatcaaaaggaccgtcgcggccccgtccgtgttcatcttcccaccgtccgacgaacagctgaagtccgggaccgcgtccgtggtgtgcctgcttaacaacttttacccgcgggaggccaaggtgcagtggaaagtcgacaacgccctgcaatccgggaactcgcaggaatcggtcactgaacaggactctaaggacagcacttattccctctcttccaccctgaccctgtccaaggcagactacgagaagcacaaggtctacgcctgtgaagtgacccaccagggactgtcctcacctgtgaccaagtcgttcaaccgcggagaatgctaatga

SEQ ID NO: 29 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 30 - protein, 330 aa, Homo sapiens [sequence omitted]

SEQ ID NO: 31 - protein, 107 aa, Homo sapiens [sequence omitted]

SEQ ID NO: 32 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 34 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0002585 A1
(43) Pub. Date: Jan. 2, 2025

(54) ACTIVATABLE BISPECIFIC ANTI-CD47 AND ANTI-PD-L1 PROTEINS AND USES THEREOF

(71) Applicant: CENTESSA PHARMACEUTICALS (UK) LIMITED, Altrincham, Cheshire (GB)

(72) Inventor: William James Jonathan FINLAY, Glasgow (GB)

(21) Appl. No.: 18/778,061

(22) Filed: Jul. 19, 2024

Related U.S. Application Data
(63) Continuation of application No. PCT/EP2023/052366, filed on Jan. 31, 2023.
(60) Provisional application No. 63/348,842, filed on Jun. 3, 2022.
(60) Provisional application No. 63/342,283, filed on May 16, 2022.
(60) Provisional application No. 63/309,759, filed on Feb. 14, 2022.
(60) Provisional application No. 63/305,003, filed on Jan. 31, 2022.

(51) Int. Cl. C07K 16/28; A61K 39/00; A61P 35/00

(52) U.S. Cl. CPC C07K 16/2827; A61P 35/00; C07K 16/2803; A61K 2039/505; C07K 2317/31; C07K 2317/55; C07K 2317/76; C07K 2317/92; C07K 2319/50

(57) ABSTRACT

Provided herein are protein molecules that specifically bind PD-L1 and also exhibit activatable specific CD47 binding in diseased tissues. Further provided herein are uses of such protein molecules to treat cancer.

### CROSS REFERENCE TO RELATED APPLICATIONS

This application is a continuation of International Application No. PCT/EP2023/052366, filed on Jan. 31, 2023, which claims the benefit of U.S. Provisional Application No. 63/348,842, filed on Jun. 3, 2022, U.S. Provisional Application No. 63/342,283, filed on May 16, 2022, U.S. Provisional Application No. 63/309,759, filed on Feb. 14, 2022, and U.S. Provisional Application No. 63/305,003, filed on Jan. 31, 2022, the contents of each of which are hereby incorporated by reference in their entirety.

### REFERENCE TO AN ELECTRONIC SEQUENCE LISTING

The contents of the electronic sequence listing (ULSL_003_04WO_SeqList_ST26.xml; Size: 38,176 bytes; and Date of Creation: Jan. 29, 2023) are herein incorporated by reference in their entirety.

### TECHNICAL FIELD

The disclosure relates to activatable bispecific proteins and treatments for cancer.

### BACKGROUND

In immune oncology therapy, few of the key drug targets are exclusively expressed in diseased tissue, with the majority also being expressed in non-diseased tissue. In addition, many drugs employed in cancer treatment employ highly potent cell-killing mechanisms of action such as antibody-dependent cellular cytotoxicity (ADCC) and antibody-dependent cellular phagocytosis (ADCP). As a result, engagement of the target by the drug in non-diseased tissue often causes unwanted side effects.

PD-L1 is a cell surface receptor that is a member of the immunoglobulin superfamily and is principally expressed on myeloid cells and regulatory T (Treg) cells in non-diseased tissues. However, PD-L1 has also been observed to be highly expressed on some cancer cells. PD-L1 binds to the membrane protein PD1. The interaction of PD-L1 with PD1 on T cells down-regulates T cell inflammatory activity, which promotes immune self-tolerance. PD-L1 is, therefore, described as an immune checkpoint. Hence, antagonistic anti-PD-L1 monoclonal antibodies that block interaction with PD1 have demonstrated the potential to act as well-tolerated immunotherapeutic agents in disease settings such as cancer, by “liberating” T cell responses from natural immune restriction. As such, PD-L1 is a drug target used to amplify the adaptive immune system's anti-cancer effects.

CD47 is also a cell surface receptor that acts as an immune checkpoint, but its effects are principally to limit the activity of the innate immune system, especially in the case of monocytes, macrophages, natural killer (NK) cells and neutrophils. This ability to minimize innate immune cell activity is multifactorial but principally involves the activation of the SIRPα receptor when bound by CD47. Activation of the SIRPα receptor minimizes ADCC and ADCP by innate immune cells. CD47-blocking agents that also provide strong Fc effector function can maximize the therapeutic potential of the innate immune system by simultaneously blocking CD47 action while promoting FcγR-mediated cell killing.

Anti-PD-L1 antibodies could be made more potent therapeutic agents by gaining the ability to also block CD47 and strongly engage Fc gamma receptors (e.g., in bispecific antibody format with an IgG1 Fc). This would combine two key checkpoint inhibitor functions that can synergistically stimulate both the innate and adaptive immune system. However, the ability to make this combination work in a single therapeutic agent structure is severely limited by the extremely broad expression of CD47 on many cell types such as red and white blood cells and endothelial cells, as well as others. This broad expression profile for CD47 leads not only to dose-limiting toxicities for CD47 binding agents with active effector function, but also to profound peripheral sink/biodistribution problems that limit the ability of such agents to achieve high enough exposure in diseased tissues to exploit the combined mechanisms. There is, therefore, a need for engineered forms of bispecific binding proteins with activity specifically targeted to the diseased tissue environment.

### SUMMARY

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6; wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises the amino acid sequence of SEQ ID NO: 21, and the anti-PD-L1 VL domain comprises the amino acid sequence of SEQ ID NO: 22; and wherein the anti-CD47 VH domain comprises the amino acid sequence of SEQ ID NO: 23, and the anti-CD47 VL domain comprises the amino acid sequence of SEQ ID NO: 24.

Provided herein is a protein comprising a heavy chain and a light chain, wherein the heavy chain comprises the amino acid sequence of SEQ ID NO: 25; and wherein the light chain comprises the amino acid sequence of SEQ ID NO: 26.

In some embodiments, the first linker comprises the amino acid sequence of SEQ ID NO: 13. In some embodiments, the second linker comprises the amino acid sequence of SEQ ID NO: 14.

In some embodiments, the immunoglobulin heavy chain constant region of a protein provided herein is an IgG, IgE, IgM, IgD, IgA, or IgY constant region. In some embodiments, the immunoglobulin heavy chain constant region of a protein provided herein is an IgG1, IgG2, IgG3, IgG4, IgA1 or IgA2 constant region. In some embodiments, the immunoglobulin heavy chain constant region of a protein provided herein is immunologically inert. In some embodiments, the immunoglobulin heavy chain constant region of a protein provided herein is a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.

Provided herein is a multimeric protein that is a dimer of two identical proteins, wherein each protein is a protein disclosed herein.

Provided herein is an immunoconjugate comprising a protein disclosed herein linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Provided herein is a pharmaceutical composition comprising a protein, a multimeric protein, or an immunoconjugate disclosed herein, and a pharmaceutically acceptable carrier, diluent or excipient.

Provided herein is a nucleic acid molecule encoding (a) the heavy chain amino acid sequence; (b) the light chain amino acid sequence; or (c) both the heavy chain and the light chain amino acid sequences of a protein disclosed herein. In some embodiments, the nucleic acid molecule comprises the nucleotide sequence of SEQ ID NO: 27, the nucleotide sequence SEQ ID NO: 28, or the nucleotide sequences of both SEQ ID NO: 27 and SEQ ID NO: 28.

Provided herein is an expression vector comprising a nucleic acid molecule disclosed herein. Provided herein is a recombinant host cell comprising a nucleic acid molecule disclosed herein or an expression vector disclosed herein.

Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Provided herein is a method for enhancing an anti-cancer immune response in a subject, the method comprising administering to the subject a therapeutically effective amount of a protein, a multimeric protein, an immunoconjugate, or a pharmaceutical composition disclosed herein.

Provided herein is a method for treating cancer in a subject, the method comprising administering to the subject a therapeutically effective amount of a protein, a multimeric protein, an immunoconjugate, or a pharmaceutical composition disclosed herein.

Provided herein is a method for ameliorating a symptom of cancer in a subject, the method comprising administering to the subject a therapeutically effective amount of a protein, a multimeric protein, an immunoconjugate, or a pharmaceutical composition disclosed herein.

Provided herein is a method for ameliorating a symptom of cancer in a subject, the method comprising administering to the subject a therapeutically effective amount of a protein, a multimeric protein, an immunoconjugate, or a pharmaceutical composition disclosed herein, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

Provided herein is a method for reducing the size of a tumor in a subject, the method comprising administering to the subject a therapeutically effective amount of a protein, a multimeric protein, an immunoconjugate, or a pharmaceutical composition disclosed herein.

Provided herein is a method for inhibiting the growth of a tumor in a subject, the method comprising administering to the subject a therapeutically effective amount of a protein, a multimeric protein, an immunoconjugate, or a pharmaceutical composition disclosed herein.

Provided herein is a protein, a multimeric protein, an immunoconjugate, or a pharmaceutical composition disclosed herein for use in enhancing an anti-cancer immune response in a subject.

Provided herein is a protein, a multimeric protein, an immunoconjugate, or a pharmaceutical composition disclosed herein for use in treating cancer in a subject.

Provided herein is a protein, a multimeric protein, an immunoconjugate, or a pharmaceutical composition disclosed herein for use in ameliorating a symptom of cancer in a subject.

Provided herein is a protein, a multimeric protein, an immunoconjugate, or a pharmaceutical composition disclosed herein for use in ameliorating a symptom of cancer in a subject, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

Provided herein is a protein, a multimeric protein, an immunoconjugate, or a pharmaceutical composition disclosed herein for use in reducing size of a tumor in a subject.

Provided herein is a protein, a multimeric protein, an immunoconjugate, or a pharmaceutical composition disclosed herein for use in inhibiting the growth of a tumor in a subject.

### BRIEF DESCRIPTION OF THE DRAWINGS

FIG. 1 depicts a diagram of a protein molecule disclosed herein (LB101) in intact (left) and protease-cleaved (right) conformations. In an intact conformation, only PD-L1 binding domains are exposed and able to bind their cognate target. Linkers in both the heavy and light chains in this design are proteolytically cleavable and may be sequentially cleaved by matrix metalloproteases (MMPs) and/or cathepsins, with a first “fast” cleavage taking intact structure and creating an intermediate protease-cleaved active state which allows both PD-L1 and CD47 Fabs from a single protein construct to bind their cognate targets.

FIG. 2 depicts a diagram of the mechanism of activity of activatable bispecific protein molecules provided herein, such as LB101 (labeled as “CD47 LockBody”).

FIG. 3A depicts an SDS-PAGE analysis of purified LB101 protein. Lane 1—Molecular weight standards. Lane 2—LB101 (reduced conditions). Lane 3—blank. Lane 4—LB101 (unreduced conditions).

FIG. 3B depicts a Size Exclusion Chromatography (SEC) analysis of purified LB101 protein, demonstrating structural uniformity.

FIG. 4 depicts an ELISA analysis of binding for LB101 to immobilized human PD-L1 or immobilized human CD47. A time course analysis was performed for hinge linker cleavage with or without human MMP12, from 0 to 24h.

FIG. 5A depicts phagocytosis of A549 cancer cells by human CD11b+ cells by IgG1 Isotype and vehicle (no phagocytosis), LB101 with no MMP12 treatment (weak phagocytosis), LB101 MMP12 treated (strong phagocytosis) and CD47 IgG4 (strong phagocytosis).

FIG. 5B depicts phagocytosis of HEL cancer cells by human CD11b+ cells by IgG1 Isotype and vehicle (no phagocytosis), LB101 with no MMP12 treatment (weak phagocytosis), LB101 MMP12 treated (strong phagocytosis) and CD47 IgG4 (strong phagocytosis).

FIG. 6A depicts hPD-L1+MC38 tumor growth in transgenic hPD-L1/hPD1 C57B6 mice. Mice were treated with either IgG1 isotype (5 mg/kg), Atezolizumab (anti-PD-L1 antibody) (5 mg/kg) or LB101 (8.5 mg/kg, molar equivalent to 5 mg/kg of an IgG1).

FIG. 6B depicts a Kaplan-Meier plot of survival for hPD-L1+MC38 tumor growth in transgenic hPD-L1/hPD1 C57B6 mice. Five groups in total were treated with either IgG1 isotype (5 mg/kg), Atezolizumab (anti-PD-L1 antibody) (5 mg/kg or 10 mg/kg) or LB101 (8.5 mg/kg or 17 mg/kg, molar equivalent to 5 mg/kg or 10 mg/kg of an IgG1).

FIG. 6C depicts hPD-L1+MC38 tumor growth in transgenic hPD-L1/hPD1 C57B6 mice. Mice were treated with either IgG1 isotype (5 mg/kg), Atezolizumab (anti-PD-L1 antibody) (10 mg/kg) or LB101 (17 mg/kg, molar equivalent to 10 mg/kg of an IgG1).

FIG. 6D depicts hPD-L1+MC38 tumor growth in individual transgenic hPD-L1/hPD1 C57B6 mice. Mice were treated with either Atezolizumab (anti-PD-L1 antibody) (5 mg/kg) or LB101 (8.5 mg/kg, molar equivalent to 5 mg/kg of an IgG1).

FIG. 6E depicts hPD-L1+MC38 tumor growth in transgenic hPD-L1/hPD1 C57B6 mice. Mice were treated with either IgG1 isotype control (5 mg/kg), or a dose titration of LB101 (0.3, 1.5, 4.5 and 8.5 mg/kg).

FIG. 7 depicts activity of IgG1 isotype, Atezolizumab (anti-PD-L1 antibody) or LB101 (before or after treatment with MMP12 for 1 or 2 hours) in a cell-based assay which measures the ability of agents to interrupt hPD-L1/hPD1 signaling (measured in fold activation).

FIG. 8 depicts hPD-L1+MC38 tumor growth during a “rechallenge” experiment in transgenic hPD-L1/hPD1 C57B6 mice. Naïve mice (n=10) and non-naïve mice that had previously exhibited complete tumor regression when treated with LB101 (8.5 mg/kg, n=13) or LB101 (17 mg/kg, n=9) were tested.

FIG. 9 depicts hPD-L1+MC38 tumor growth during a “rechallenge” experiment in transgenic hPD-L1/hPD1 C57B6 mice, over 30 days. Naïve mice (n=10) and non-naïve mice that had previously exhibited complete tumor regression when treated with LB101 (8.5 mg/kg, n=13) or LB101 (17 mg/kg, n=9) were tested.

FIG. 10 depicts immune cell infiltrates in the hPD-L1+MC38 tumor, as measured by flow cytometry. Tumors (n=8/group) were isolated from mice treated with human IgG1 isotype, Atezolizumab (anti-PD-L1 antibody) or LB101. Numbers in each analysis are reported as % of total tumor CD45+ (immune) cells. Significance values (p<0.05) for difference between groups were calculated and are shown as line and asterisk.

FIG. 11A-FIG. 11F depict results from a dose range finding (DRF) study of LB101 in cynomolgus monkeys. Male (m) and Female (f) monkeys were dosed four times, once every seven days, at 5, 20 or 50 mg/kg of LB101. Blood samples were taken and measurements were made of LB101 pharmacokinetics (FIG. 11A), Body weight (FIG. 11B), Red Blood Cell levels (RBC, FIG. 11C), Hemoglobin levels (HBG, FIG. 11D), Platelet levels (FIG. 11E) and Neutrophil levels (FIG. 11F). Windows for normal levels are shown as grey boxes.

### DETAILED DESCRIPTION

Provided herein are proteins that are conditionally active in diseased human tissues. The proteins of the disclosure are fully active in specifically binding and blocking PD-L1 throughout the body; exhibit minimized binding of CD47 in healthy tissue; and become highly activated in CD47 binding and blocking once in the diseased tissue environment. A protein of the disclosure comprises a CD47 binding domain that is masked by a PD-L1 binding domain in non-diseased tissues. The protein also comprises two peptide linkers that are cleaved by one or more proteases expressed in a diseased tissue (e.g, a tumor). The linker cleavage unmasks the CD47 binding domain in the diseased tissue, thus allowing binding and/or function of the protein selectively in the diseased tissue.

### Protein Molecules

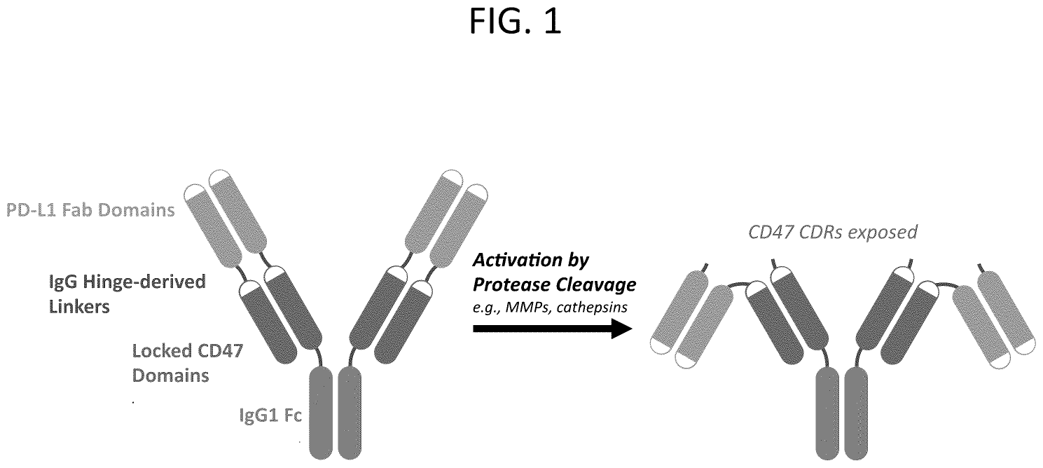

Provided herein are proteins comprising a heavy chain and a light chain, wherein the heavy chain comprises, in N-terminus to C-terminus order, an anti-PD-L1 heavy chain variable (VH) domain, a CH domain, a first linker, an anti-CD47 VH domain, and an immunoglobulin heavy chain constant region; and wherein the light chain comprises, in N-terminus to C-terminus order, an anti-PD-L1 light chain variable (VL) domain, a first immunoglobulin light chain constant region, a second linker, an anti-CD47 VL domain, and a second immunoglobulin light chain constant region. The first linker and the second linker are cleavable by matrix metalloproteases (MMPs) and/or cathepsins found in diseased tissues, such as tumors. In some embodiments, the immunoglobulin heavy chain constant region comprises, in N-terminus to C-terminus order, a CH1 domain, a hinge, a CH2 domain, and a CH3 domain. A diagram of a protein of the disclosure, with labeled domains, is shown in FIG. 1.

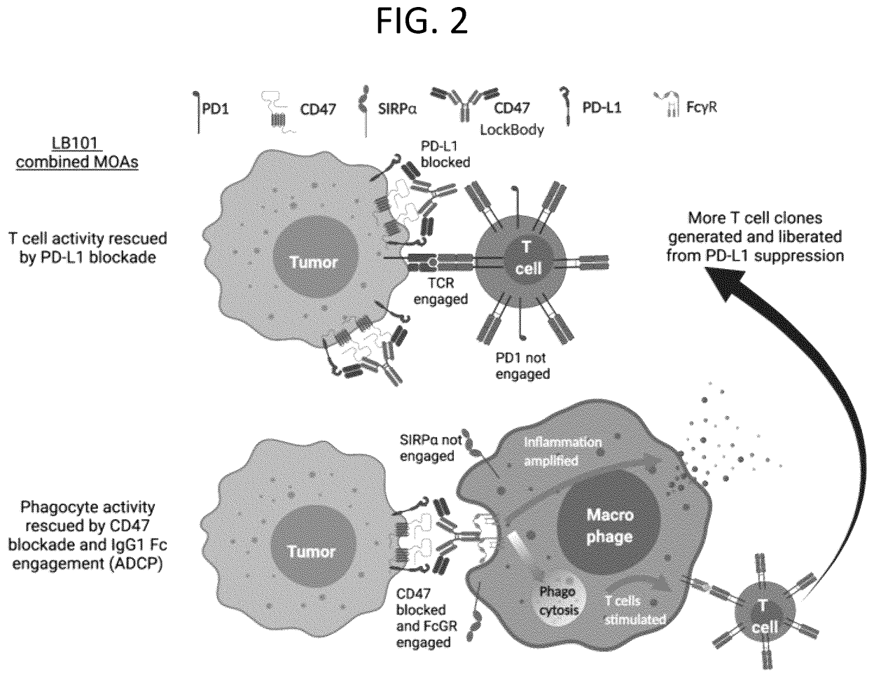

The anti-PD-L1/anti-CD47 protein structure shown in FIG. 1 minimizes binding to CD47 outside of diseased tissue. This effect is achieved by adding PD-L1 and linkers above (N-terminal to) the CD47 binding domains. The use of appropriate upper domain/linker combinations results in a configuration that fully blocks binding activity in the lower CD47 binding domain. The PD-L1 domain then drives high concentration in PD-L1-enriched tumor microenvironments. The protein construct linker system exploits the elevated MMP and cathepsin activity that is common in solid tumors to cleave the linker peptides, exposing the CD47 binding domains and thereby conditionally activating the CD47-binding activity in the tumor, rather than the periphery. The combined biological functions of proteins provided herein thereby afford the molecule the potential to augment both innate and adaptive immune responses to cancer cells, as outlined in FIG. 2.

The protein design disclosed herein may be based on sequences derived from IgG1, IgG2, IgG3, IgG4, IgE, IgM, or IgA and may or may not have effector function capacity. In the construct shown in FIG. 1, four polypeptide chains encode for four Fab domains (2× Fab A, 2× Fab B), four linker sequences, and may or may not have an immunoglobulin hinge region and an Fc domain. The linkers in the protein design are both proteolytically cleavable and may be sequentially cleaved, with a first “fast” cleavage taking the intact structure and creating an intermediate active state which allows Fabs A and B from a single protein construct to bind their cognate targets. Cleaved linkers derived from immunoglobulin hinge sequences may also recruit increased immune effector function (ADCC, CDC and/or ADCP) at the cell membrane via endogenous anti-hinge antibodies.

Proteins disclosed herein comprise domains and regions of antibody molecules. The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally, IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

As used herein, the terms “immunological binding” and “immunological binding properties” refer to the non-covalent interactions of the type which occur between an immunoglobulin molecule (e.g., antibody or antigen-binding portion thereof), or a protein comprising an immunoglobulin-derived binding domain(s) and an antigen for which the immunoglobulin or protein is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind PD-L1 or CD47 when the equilibrium binding constant (K_{d}) is ≤10 μM, preferably ≤10 nM, more preferably ≤10 nM, and most preferably ≤100 μM to about 1 μM, as measured by assays such as radioligand binding assays or similar assays known to those skilled in the art. One method for determining the K_{d} of an antibody is by using surface plasmon resonance (SPR), typically using a biosensor system such as a Biacore® system.

Functionally, the binding affinity of a protein provided herein may be within the range of 10⁻⁵M to 10⁻¹² M. For example, the binding affinity of a protein provided herein is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵M to 10⁻¹⁰ M, from 10⁻⁶ M to 10⁻¹⁰M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region. In some embodiments, the first linker is the same as the second linker. In some embodiments, the first linker is not the same as the second linker. In some embodiments, the first linker comprises an amino acid sequence selected from any one of SEQ ID NOs: 13-20. In some embodiments, the second linker comprises an amino acid sequence selected from any one of SEQ ID NOs: 13-20. In some embodiments, the first linker comprises the amino acid sequence of SEQ ID NO: 13. In some embodiments, the second linker comprises the amino acid sequence of SEQ ID NO: 14. In some embodiments, the first linker comprises the amino acid sequence of SEQ ID NO: 13; and the second linker comprises the amino acid sequence of SEQ ID NO: 14.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises at least one HCDR sequence selected from: a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VL domain comprises at least one LCDR sequence selected from: a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-CD47 VH domain comprises at least one HCDR sequence selected from: a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-CD47 VL domain comprises at least one LCDR sequence selected from: a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6; wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6; wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; wherein the first linker comprises the amino acid sequence of SEQ ID NO: 13; and wherein the second linker comprises the amino acid sequence of SEQ ID NO: 14.

Further provided herein is a protein comprising a heavy chain and a light chain, wherein the heavy chain comprises, in N-terminus to C-terminus order, an anti-PD-L1 heavy chain VH domain, a CH domain, a first linker, an anti-CD47 VH domain, and an immunoglobulin heavy chain constant region; wherein the light chain comprises, in N-terminus to C-terminus order, an anti-PD-L1 VL domain, a first immunoglobulin light chain constant region, a second linker, an anti-CD47 VL domain, and a second immunoglobulin light chain constant region; wherein the anti-PD-L1 VH domain comprises the amino acid sequence of SEQ ID NO: 21, and the anti-PD-L1 VL domain comprises the amino acid sequence of SEQ ID NO: 22; and wherein the anti-CD47 VH domain comprises the amino acid sequence of SEQ ID NO: 23, and the anti-CD47 VL domain comprises the amino acid sequence of SEQ ID NO: 24.

Also provided herein is a protein comprising a heavy chain and a light chain, wherein the heavy chain comprises the amino acid sequence of SEQ ID NO: 25; and wherein the light chain comprises the amino acid sequence of SEQ ID NO: 26. The protein comprising these heavy chain and light chain sequences is referred to as LB101 or LB-101. In some embodiments, a protein comprises one or more amino acid sequences provided in Table 1.

Further provided herein is a protein comprising a heavy chain and a light chain, wherein the heavy chain comprises an amino acid sequence that is at least 90%, 91%, 92%, 93%, 94%, 95%, 96%, 97%, 98% or 99% identical to the amino acid sequence of SEQ ID NO: 25; and wherein the light chain comprises an amino acid sequence that is at least 90%, 91%, 92%, 93%, 94%, 95%, 96%, 97%, 98% or 99% identical to the amino acid sequence of SEQ ID NO: 26.

Also provided herein is a protein comprising a heavy chain and a light chain, wherein the heavy chain comprises the amino acid sequence of SEQ ID NO: 25, with 1, 2 or 3 conservative amino acid substitutions; and wherein the light chain comprises the amino acid sequence of SEQ ID NO: 26, with 1, 2 or 3 conservative amino acid substitutions. In some embodiments, conservative amino acid substitutions are made only in the FR sequences and not in the CDR sequences. In some embodiments, conservative amino acid substitutions are not made in the first linker or the second linker sequences.

TABLE 1
Exemplary sequences

| Domain or Region | Sequence | SEQ ID NO |
|---|---|---|
| Anti-PD-L1 HCDR1 | TYAIS | 1 |
| Anti-PD-L1 HCDR2 | GIIPIFGKAHYAQKFQG | 2 |
| Anti-PD-L1 HCDR3 | KFHFVSGSPFGMDV | 3 |
| Anti-PD-L1 LCDR1 | RASQSVSSYLA | 4 |
| Anti-PD-L1 LCDR2 | DASNRAT | 5 |
| Anti-PD-L1 LCDR3 | QQRSNWPT | 6 |
| Anti-CD47 HCDR1 | NYYIF | 7 |
| Anti-CD47 HCDR2 | IINPVDGDTNYNPSFQG | 8 |
| Anti-CD47 HCDR3 | GGYTMDR | 9 |
| Anti-CD47 LCDR1 | RSSQSLLHSNGYNYLH | 10 |
| Anti-CD47 LCDR2 | KVSNRFS | 11 |
| Anti-CD47 LCDR3 | FQNTHTPRT | 12 |
| Linker LHL | GPAPELLGGGS | 13 |
| Linker LHL-F | GPAPLGLGGGS | 14 |
| Linker L1 | GGGGS | 15 |
| Linker L2 | GGGGSGGGGS | 16 |
| Linker L3 | GGGGSGGGGSGGGGS | 17 |
| Linker LH | GPAPELL | 18 |
| Linker LHL-M | PPCPAPELLGGGS | 19 |
| Linker LHL-MF | PPCPAPLGLGGGS | 20 |
| Anti-PD-L1 VH | QVQLVQSGAEVKKPGSSVKVSCKTSGDTFSTYAISWV | 21 |
| domain (amino acid) | RQAPGQGLEWMGGIIPIFGKAHYAQKFQGRVTITADE | |
| | STSTAYMELSSLRSEDTAVYFCARKFHFVSGSPFGMD | |
| | VWGQGTTVTVSS | |
| Anti-PD-L1 VL | EIVLTQSPATLSLSPGERATLSCRASQSVSSYLAWYQ | 22 |
| domain (amino acid) | QKPGQAPRLLIYDASNRATGIPARFSGSGSGTDFTLT | |
| | ISSLEPEDFAVYYCQQRSNWPTFGQGTKVEIK | |
| Anti-CD47 VH | QVQLVQSGAEVKKPGESLKISCKGSGYTFTNYYIFWV | 23 |
| domain (amino acid) | RQMPGKGLEWMGIINPVDGDTNYNPSFQGQVTISADK | |
| | SISTAYLQWSSLKASDTAMYYCARGGYTMDRWGQGTL | |
| | VTVSS | |
| Anti-CD47 VL | DIVMTQSPLSLPVTPGEPASISCRSSQSLLHSNGYNY | 24 |
| domain (amino acid) | LHWYLQKPGQSPQLLIYKVSNRFSGVPDRFSGSGSGT | |
| | DFTLKISRVEAEDVGVYYCFQNTHTPRTFGGGTKVEI | |
| | K | |
| LB101 Heavy chain | QVQLVQSGAEVKKPGSSVKVSCKTSGDTFSTYAISWV | 25 |
| (amino acid) | RQAPGQGLEWMGGIIPIFGKAHYAQKFQGRVTITADE | |
| | STSTAYMELSSLRSEDTAVYFCARKFHFVSGSPFGMD | |
| | VWGQGTTVTVSSASTKGPSVFPLAPSSKSTSGGTAAL | |
| | GCLVKDYFPEPVTVSWNSGALTSGVHTFPAVLQSSGL | |
| | YSLSSVVTVPSSSLGTQTYICNVNHKPSNTKVDKKVE | |
| | PKSCGPAPELLGGGSQVQLVQSGAEVKKPGESLKISC | |
| | KGSGYTFTNYYIFWVRQMPGKGLEWMGIINPVDGDTN | |
| | YNPSFQGQVTISADKSISTAYLQWSSLKASDTAMYYC | |
| | ARGGYTMDRWGQGTLVTVSSASTKGPSVFPLAPSSKS | |
| | TSGGTAALGCLVKDYFPEPVTVSWNSGALTSGVHTFP | |
| | AVLQSSGLYSLSSVVTVPSSSLGTQTYICNVNHKPSN | |
| | TKVDKKVEPKSCDKTHTCPPCPAPELLGGPSVFLFPP | |
| | KPKDTLMISRTPEVTCVVVDVSHEDPEVKENWYVDGV | |
| | EVHNAKTKPREEQYNSTYRVVSVLTVLHQDWLNGKEY | |
| | KCKVSNKALPAPIEKTISKAKGQPREPQVYTLPPSRD | |
| | ELTKNQVSLTCLVKGFYPSDIAVEWESNGQPENNYKT | |
| | TPPVLDSDGSFFLYSKLTVDKSRWQQGNVFSCSVMHE | |
| | ALHNHYTQKSLSLSPGK | |
| LB101 Light chain | EIVLTQSPATLSLSPGERATLSCRASQSVSSYLAWYQ | 26 |
| (amino acid) | QKPGQAPRLLIYDASNRATGIPARFSGSGSGTDFTLT | |
| | ISSLEPEDFAVYYCQQRSNWPTFGQGTKVEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGECGPAPLGLGG | |
| | GSDIVMTQSPLSLPVTPGEPASISCRSSQSLLHSNGY | |
| | NYLHWYLQKPGQSPQLLIYKVSNRFSGVPDRFSGSGS | |
| | GTDFTLKISRVEAEDVGVYYCFQNTHTPRTFGGGTKV | |
| | EIKRTVAAPSVFIFPPSDEQLKSGTASVVCLLNNFYP | |
| | REAKVQWKVDNALQSGNSQESVTEQDSKDSTYSLSST | |
| | LTLSKADYEKHKVYACEVTHQGLSSPVTKSENRGEC | |
| LB101 Heavy chain | ATGGGATGGACCCTCGTGTTCCTCTTTCTGTTGTCCG | 27 |
| (nucleotide) | TGACCGCCGGAGTCCACAGCCAAGTCCAACTGGTGCA | |
| (includes sequence | GTCCGGTGCCGAAGTGAAAAAGCCCGGATCCTCGGTG | |
| encoding for leader | AAAGTGTCGTGCAAGACCTCGGGAGACACTTTCTCGA | |
| peptide) | CCTACGCCATTAGCTGGGTCAGACAGGCCCCGGGCCA | |
| | GGGCCTGGAATGGATGGGTGGAATTATCCCCATTTTC | |
| | GGAAAGGCCCACTACGCGCAGAAATTCCAGGGCCGGG | |
| | TCACCATTACTGCCGATGAGAGCACCTCGACTGCCTA | |
| | CATGGAACTGTCGTCGCTGAGGTCCGAGGATACCGCC | |
| | GTGTACTTCTGCGCCCGGAAATTCCATTTCGTGAGCG | |
| | GTTCGCCGTTCGGAATGGATGTCTGGGGACAGGGCAC | |
| | TACCGTGACCGTGAGCAGCGCCTCCACAAAGGGGCCA | |
| | TCCGTATTCCCACTTGCTCCTTCTAGCAAGTCCACAT | |
| | CAGGTGGGACAGCAGCTCTGGGCTGTCTCGTGAAGGA | |
| | CTACTTTCCTGAACCTGTTACTGTGTCCTGGAATAGC | |
| | GGCGCCTTGACCTCTGGCGTACATACCTTTCCAGCAG | |
| | TGCTCCAGTCCTCAGGGCTGTATTCTCTGTCTAGTGT | |
| | TGTAACCGTGCCCAGTTCTAGCTTGGGTACTCAGACC | |
| | TACATCTGCAATGTGAACCACAAGCCCTCAAACACAA | |
| | AAGTCGACAAGAAGGTGGAGCCTAAATCCTGCGGGCC | |
| | AGCCCCCGAGCTGCTTGGAGGAGGGTCCCAAGTGCAG | |
| | CTCGTGCAGAGCGGGGCTGAGGTCAAGAAGCCTGGAG | |
| | AGTCCCTGAAGATCTCCTGCAAGGGAAGCGGATACAC | |
| | CTTCACCAACTACTATATCTTTTGGGTGCGCCAGATG | |
| | CCGGGAAAGGGACTCGAGTGGATGGGCATCATTAACC | |
| | CGGTGGACGGCGACACTAACTACAACCCGAGCTTCCA | |
| | AGGACAAGTGACCATCTCAGCGGACAAGTCCATTTCC | |
| | ACTGCGTACCTTCAATGGTCATCGCTTAAGGCGTCGG | |
| | ACACGGCTATGTACTACTGTGCAAGAGGAGGATACAC | |
| | CATGGATCGCTGGGGCCAGGGCACTCTTGTGACCGTG | |
| | TCATCAGCGTCCACCAAGGGTCCCTCCGTGTTCCCTC | |
| | TCGCGCCGTCCTCAAAGTCTACCTCCGGTGGAACTGC | |
| | CGCGCTCGGTTGTCTCGTGAAGGACTACTTCCCGGAG | |
| | CCTGTGACTGTCTCCTGGAACTCCGGGGCCCTCACCA | |
| | GCGGAGTGCACACTTTCCCCGCCGTGCTGCAATCCTC | |
| | CGGCCTGTACAGCCTGTCCTCCGTCGTGACTGTGCCT | |
| | AGCTCCTCCCTGGGAACCCAGACCTACATCTGCAACG | |
| | TGAACCACAAGCCCTCCAACACCAAGGTCGACAAGAA | |
| | GGTCGAACCGAAGTCGTGCGACAAGACTCATACGTGC | |
| | CCTCCTTGCCCGGCCCCGGAACTGCTGGGAGGCCCAT | |
| | CCGTGTTCCTGTTCCCACCCAAGCCTAAGGATACCCT | |
| | GATGATCAGCAGAACACCGGAAGTGACCTGTGTGGTG | |
| | GTGGACGTCAGCCACGAAGATCCCGAGGTCAAGTTCA | |
| | ATTGGTACGTGGACGGGGTGGAGGTGCACAACGCAAA | |
| | GACCAAGCCCCGGGAGGAACAGTACAACTCCACCTAT | |
| | CGCGTGGTGTCGGTGCTGACGGTGCTGCACCAGGACT | |
| | GGTTGAACGGAAAGGAGTATAAGTGCAAAGTGTCGAA | |
| | CAAGGCCCTGCCCGCTCCTATCGAAAAGACCATCTCC | |
| | AAGGCCAAGGGCCAGCCGCGGGAACCCCAGGTCTACA | |
| | CTCTCCCACCGAGCCGCGACGAACTGACTAAGAATCA | |
| | AGTGTCGCTGACTTGCCTCGTCAAGGGCTTCTACCCG | |
| | TCCGACATCGCCGTGGAATGGGAGAGCAACGGCCAGC | |
| | CGGAAAACAACTACAAGACCACCCCTCCCGTGCTGGA | |
| | TTCCGACGGGTCCTTCTTCCTGTACTCAAAACTGACC | |
| | GTGGATAAGTCCAGATGGCAGCAGGGCAATGTCTTTT | |
| | CATGCTCCGTGATGCACGAGGCTCTGCATAACCACTA | |
| | CACCCAGAAGTCGCTGTCCCTGTCCCCGGGGTAATGA | |
| LB101 Light chain | ATGGTGTCCTCCGCTCAATTCCTCGGACTTCTTCTCC | 28 |
| (nucleotide) | TGTGTTTCCAAGGCACCCGCTGTGAAATCGTGCTGAC | |
| (includes sequence | TCAGTCACCCGCCACTCTGTCACTTTCACCCGGGGAG | |
| encoding for leader | AGGGCCACTCTGAGCTGCCGGGCCAGCCAGAGCGTGT | |
| peptide) | CCAGTTACCTGGCCTGGTACCAGCAAAAGCCCGGACA | |
| | GGCCCCGAGACTGCTGATCTACGATGCATCCAACCGC | |
| | GCCACTGGTATTCCTGCCCGGTTTAGCGGCTCCGGTT | |
| | CCGGGACCGACTTCACTCTCACCATTTCCTCCCTGGA | |
| | ACCGGAAGATTTCGCCGTGTACTACTGCCAGCAGCGA | |
| | TCCAACTGGCCAACGTTTGGACAGGGAACCAAGGTAG | |
| | AGATCAAACGGACCGTCGCTGCCCCTTCGGTATTCAT | |
| | CTTTCCGCCTTCGGACGAACAGTTGAAAAGCGGAACA | |
| | GCCTCGGTCGTGTGCTTGCTCAACAACTTCTATCCCA | |
| | GAGAGGCGAAAGTCCAGTGGAAGGTAGACAACGCTCT | |
| | CCAGTCAGGAAACTCGCAAGAGAGCGTAACCGAACAG | |
| | GATTCGAAGGACTCCACCTACTCCCTGTCGAGCACGC | |
| | TGACACTCTCAAAAGCGGATTATGAGAAACATAAAGT | |
| | ATATGCATGCGAGGTCACGCACCAGGGTCTGTCCTCG | |
| | CCCGTGACGAAAAGCTTCAATCGCGGCGAGTGCGGAC | |
| | CGGCCCCGCTGGGCCTGGGCGGTGGATCCGACATTGT | |
| | GATGACCCAATCTCCCCTGAGCTTACCTGTGACCCCT | |
| | GGGGAACCAGCGAGCATTAGCTGCAGATCCAGCCAGT | |
| | CCCTGCTGCACTCAAACGGATACAATTATCTGCATTG | |
| | GTACCTCCAGAAGCCCGGCCAGTCCCCCCAACTCCTG | |
| | ATCTACAAGGTGTCCAACCGGTTCTCCGGAGTGCCGG | |
| | ACCGGTTCTCGGGCTCGGGCTCCGGCACTGACTTCAC | |
| | CTTGAAGATCTCGCGGGTGGAAGCTGAAGATGTCGGC | |
| | GTTTACTACTGTTTCCAAAACACCCACACCCCACGCA | |
| | CTTTCGGAGGCGGAACTAAGGTCGAGATCAAAAGGAC | |
| | CGTCGCGGCCCCGTCCGTGTTCATCTTCCCACCGTCC | |
| | GACGAACAGCTGAAGTCCGGGACCGCGTCCGTGGTGT | |
| | GCCTGCTTAACAACTTTTACCCGCGGGAGGCCAAGGT | |
| | GCAGTGGAAAGTCGACAACGCCCTGCAATCCGGGAAC | |
| | TCGCAGGAATCGGTCACTGAACAGGACTCTAAGGACA | |
| | GCACTTATTCCCTCTCTTCCACCCTGACCCTGTCCAA | |
| | GGCAGACTACGAGAAGCACAAGGTCTACGCCTGTGAA | |
| | GTGACCCACCAGGGACTGTCCTCACCTGTGACCAAGT | |
| | CGTTCAACCGCGGAGAATGCTAATGA | |
| Human CH1 domain | ASTKGPSVFPLAPSSKSTSGGTAALGCLVKDYFPEPV | 29 |
| | TVSWNSGALTSGVHTFPAVLQSSGLYSLSSVVTVPSS | |
| | SLGTQTYICNVNHKPSNTKVDKKVEPKSC | |
| Human CH1-hinge- | ASTKGPSVFPLAPSSKSTSGGTAALGCLVKDYFPEPV | 30 |
| CH2—CH3 sequence | TVSWNSGALTSGVHTFPAVLQSSGLYSLSSVVTVPSS | |
| (IgG1 wild type) | SLGTQTYICNVNHKPSNTKVDKKVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPGK | |
| Human Cₖₐₚₚₐ domain | RTVAAPSVFIFPPSDEQLKSGTASVVCLLNNFYPREA | 31 |
| (Ck) | KVQWKVDNALQSGNSQESVTEQDSKDSTYSLSSTLTL | |
| | SKADYEKHKVYACEVTHQGLSSPVTKSFNRGEC | |
| Human IgG1-3M | ASTKGPSVFPLAPSSKSTSGGTAALGCLVKDYFPEPV | 32 |
| | TVSWNSGALTSGVHTFPAVLQSSGLYSLSSVVTVPSS | |
| | SLGTQTYICNVNHKPSNTKVDKKVEPKSCDKTHTCPP | |
| | CPAPEAAGAPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPGK | |
| Human IgG2 wild | ASTKGPSVFPLAPCSRSTSESTAALGCLVKDYFPEPV | 33 |
| type | TVSWNSGALTSGVHTFPAVLQSSGLYSLSSVVTVPSS | |
| | NFGTQTYTCNVDHKPSNTKVDKTVERKCCVECPPCPA | |
| | PPVAGPSVFLFPPKPKDTLMISRTPEVTCVVVDVSHE | |
| | DPEVQFNWYVDGVEVHNAKTKPREEQFNSTFRVVSVL | |
| | TVVHQDWLNGKEYKCKVSNKGLPAPIEKTISKTKGQP | |
| | REPQVYTLPPSREEMTKNQVSLTCLVKGFYPSDISVE | |
| | WESNGQPENNYKTTPPMLDSDGSFFLYSKLTVDKSRW | |
| | QQGNVFSCSVMHEALHNHYTQKSLSLSPGK | |
| Human IgG4 wild | ASTKGPSVFPLAPCSRSTSESTAALGCLVKDYFPEPV | 34 |
| type | TVSWNSGALTSGVHTFPAVLQSSGLYSLSSVVTVPSS | |
| | SLGTKTYTCNVDHKPSNTKVDKRVESKYGPPCPSCPA | |
| | PEFLGGPSVFLFPPKPKDTLMISRTPEVTCVVVDVSQ | |
| | ED PEVQFNWYVDGVEVHNAKTKPREEQFNSTYRVVS | |
| | VLTVLHQDWLNGKEYKCKVSNKGLPSSIEKTISKAKG | |
| | QPREPQVYTLPPSQEEMTKNQVSLTCLVKGFYPSDIA | |
| | VEWESNGQPENNYKTTPPVLDSDGSFFLYSRLTVDKS | |
| | RWQEGNVFSCSVMHEALHNHYTQKSLSLSLGK | |
| Human IgG4(S228P) | ASTKGPSVFPLAPCSRSTSESTAALGCLVKDYFPEPV | 35 |
| | TVSWNSGALTSGVHTFPAVLQSSGLYSLSSVVTVPSS | |
| | SLGTKTYTCNVDHKPSNTKVDKRVESKYGPPCPPCPA | |
| | PEFLGGPSVFLFPPKPKDTLMISRTPEVTCVVVDVSQ | |
| | ED PEVQFNWYVDGVEVHNAKTKPREEQFNSTYRVVS | |
| | VLTVLHQDWLNGKEYKCKVSNKGLPSSIEKTISKAKG | |
| | QPREPQVYTLPPSQEEMTKNQVSLTCLVKGFYPSDIA | |
| | VEWESNGQPENNYKTTPPVLDSDGSFFLYSRLTVDKS | |
| | RWQEGNVFSCSVMHEALHNHYTQKSLSLSLGK | |

In the VH and VL domain sequences, the CDR sequences are underlined. In the heavy
and light chain amino acid sequences, the anti-PD-L1 VH and VL domain sequences
are italicized and underlined; the cleavable linker sequences are underlined, and
the anti-CD47 VH and VL domain sequences are in bold font.

In some embodiments, a protein provided herein comprises at least one Fab fragment, which is a monovalent antigen-binding fragment consisting of the VL, VH, CL and CH1 domains.

In some embodiments, a protein provided herein comprises an immunoglobulin heavy chain constant region. In some embodiments, the immunoglobulin heavy chain constant region is IgG, IgE, IgM, IgD, IgA or IgY. In some embodiments, the immunoglobulin heavy chain constant region is IgG1, IgG2, IgG3, IgG4, IgA1 or IgA2. In some embodiments, the immunoglobulin heavy chain constant region is IgG1. In some embodiments, the immunoglobulin heavy chain constant region is immunologically inert. In some embodiments, the immunoglobulin heavy chain constant region comprises one or more mutations to reduce or prevent FcγR binding, antibody-dependent cell-mediated cytotoxicity (ADCC) activity, antibody-dependent cellular phagocytosis (ADCP), and/or complement-dependent cytotoxicity (CDC) activity. In some embodiments, the immunoglobulin heavy chain constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kabat. In some embodiments, a position of an amino acid residue in a constant region of an immunoglobulin molecule is numbered according to the EU index as in Kabat (Ward et al., 1995 Therap. Immunol. 2:77-94).

In some embodiments, a protein provided herein may comprise an immunoglobulin light chain constant region that is a kappa light chain. In some embodiments a kappa light chain comprises SEQ ID NO: 31.

In some embodiments, a protein provided herein may comprise an immunoglobulin light chain constant region that is a lambda light chain.

In some embodiments, a protein provided herein may comprise an immunoglobulin heavy chain constant region comprising an amino acid sequence of an Fc region of human IgG4, human IgG4(S228P), human IgG2, human IgG1, human IgG1 effector null. For example, the human IgG4(S228P) Fc region comprises the following substitution compared to the wild-type human IgG4 Fc region: S228P. For example, the human IgG1 effector null Fc region comprises the following substitutions compared to the wild-type human IgG1 Fc region: L234A, L235A and G237A. In some embodiments, a protein may comprise an immunoglobulin heavy chain constant region comprising the amino acid sequence of any one of SEQ ID NO: 29, SEQ ID NO: 30, SEQ ID NO: 32, SEQ ID NO: 33, SEQ ID NO: 34 and SEQ ID NO: 35.

Further provided herein is a multimeric protein that is a dimer of two identical proteins, wherein each protein is a protein disclosed herein. FIG. 1 shows an example of a such a dimer. The dimer comprises two identical heavy chains and two identical light chains. In some embodiments, each heavy chain comprises the amino acid sequence of SEQ ID NO: 25, and each light chain comprises the amino acid sequence of SEQ ID NO: 26.

Provided herein is an immunoconjugate comprising a protein disclosed herein linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example, RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

### Pharmaceutical Compositions

The activatable proteins provided herein (also referred to herein as “active compounds”) can be incorporated into pharmaceutical compositions suitable for administration. Such compositions typically comprise a protein (or an immunoconjugate comprising said protein, or a multimeric protein comprising said protein), and a pharmaceutically acceptable carrier, diluent or excipient. Such materials should be non-toxic and should not interfere with the efficacy of the protein. The precise nature of the carrier or other material will depend on the route of administration, which may be by injection, bolus, infusion, or any other suitable route, as discussed below. Provided herein is a composition comprising a protein (or an immunoconjugate comprising said protein, or a multimeric protein comprising said protein) disclosed herein, and a pharmaceutically acceptable carrier.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the protein, an increase in its lifespan and/or in its efficacy in the body or an increase in its solubility in solution.

In all embodiments of the pharmaceutical compositions provided herein, the protein may comprise a heavy chain and a light chain, wherein the heavy chain comprises the amino acid sequence of SEQ ID NO: 25; and wherein the light chain comprises the amino acid sequence of SEQ ID NO: 26.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). The composition can be sterile; can be fluid to the extent that easy syringeability exists; can be stable under the conditions of manufacture and storage; and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the protein may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Antibodies

Provided herein is a nucleic acid molecule (e.g., an isolated nucleic acid molecule) encoding an amino acid sequence of a protein disclosed herein (or an amino acid sequence of a (i) VH domain, (ii) a VL domain, or (iii) both a VH domain and a VL domain of a protein). Further provided herein is a nucleic acid molecule (e.g., an isolated nucleic acid molecule) encoding (i) a heavy chain, (ii) a light chain, or (iii) both a heavy chain and a light chain of a protein disclosed herein. In some embodiments, a nucleic acid molecule encoding a VH domain, a VL domain, a heavy chain or a light chain comprises a signal sequence (or encodes a leader peptide). In some embodiments, a nucleic acid molecule encoding a VH domain, a VL domain, a heavy chain or a light chain does not comprise a signal sequence (or does not encode a leader peptide).

In some embodiments, the nucleic acid molecule comprises the nucleotide sequence of SEQ ID NO: 27, the nucleotide sequence SEQ ID NO: 28, or the nucleotide sequences of both SEQ ID NO: 27 and SEQ ID NO: 28, with or without the sequence encoding for the leader peptide.

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a heavy chain and a light chain of a protein disclosed herein. In some embodiments, a recombinant host cell comprises (i) a first vector or a first nucleic acid molecule encoding a heavy chain of a protein disclosed herein and (ii) a second vector or a second nucleic acid molecule encoding a light chain of a protein disclosed herein.

Antibody molecules of the invention, or antigen-binding portion thereof, can be produced using techniques well known in the art, for example, recombinant technologies, phage display technologies, synthetic technologies, computational technologies or combinations of such technologies or other technologies readily known in the art.

Further provided herein is a method for producing a protein disclosed herein, the method comprising: culturing a recombinant host cell comprising an expression vector described herein under conditions whereby the nucleic acid segment is expressed, thereby producing the protein. The protein may then be isolated from the host cell or culture. Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Proteins disclosed herein can be produced by any of a variety of methods known to those skilled in the art. In certain embodiments, proteins disclosed herein can be produced recombinantly. For example, nucleic acid sequences encoding one or more of SEQ ID NO: 25 and SEQ ID NO: 26, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, light chain proteins and heavy chain proteins are produced in a cell with a signal sequence that is removed upon production of a mature protein disclosed herein.

Those skilled in the art will be able to determine whether a protein comprising a given polypeptide sequence binds to PD-L1 protein and/or CD47 protein using standard methodologies, for example, Western blots, ELISA, and the like.

### Medical Uses of Activatable Proteins

Provided herein are methods and uses of activatable proteins, multimeric proteins, immunoconjugates, and pharmaceutical compositions disclosed herein for providing a therapeutic benefit to a subject with cancer.

An activatable protein, multimeric protein, immunoconjugate, or pharmaceutical composition disclosed herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the protein, multimeric protein, immunoconjugate, or pharmaceutical composition to a subject in need thereof.

Provided herein is a method for enhancing an anti-cancer immune response in a subject, the method comprising administering to the subject a therapeutically effective amount of a protein, a multimeric protein, an immunoconjugate, or a pharmaceutical composition disclosed herein. In some embodiments, an anti-cancer immune response is a T cell response. In some embodiments, an anti-cancer immune response is a complement response.

Provided herein is a method for treating cancer in a subject, the method comprising administering to the subject a therapeutically effective amount of a protein, a multimeric protein, an immunoconjugate, or a pharmaceutical composition disclosed herein. Provided herein is a method for ameliorating a symptom of cancer in a subject, the method comprising administering to the subject a therapeutically effective amount of a protein, a multimeric protein, an immunoconjugate, or a pharmaceutical composition disclosed herein.

Provided herein is a method for reducing the size of a tumor in a subject, the method comprising administering to the subject a therapeutically effective amount of a protein, a multimeric protein, an immunoconjugate, or a pharmaceutical composition disclosed herein.

Provided herein is a method for inhibiting the growth of a tumor in a subject, the method comprising administering to the subject a therapeutically effective amount of a protein, a multimeric protein, an immunoconjugate, or a pharmaceutical composition disclosed herein.

In some embodiments, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

In some embodiments, the cancer is a solid tumor. In some embodiments, a tumor is a gastrointestinal tumor, pancreatic tumor, breast tumor, lung tumor, bronchial tumor, colorectal tumor, prostate tumor, stomach tumor, ovarian tumor, urinary bladder tumor, brain tumor, central nervous system tumor, peripheral nervous system tumor, esophageal tumor, cervical tumor, uterine tumor, endometrial tumor, tumor of the oral cavity or pharynx, liver tumor, kidney tumor, renal tumor, testicular tumor, biliary tract tumor, small bowel tumor, appendix tumor, salivary gland tumor, thyroid tumor, or adrenal gland tumor.

In some embodiments, the cancer is a hematological cancer.

In some embodiments, a cancer of hematological tissues is a lymphoma. In some embodiments, the cancer is mantle cell lymphoma, acute lymphoblastic leukemia, chronic lymphocytic leukemia, Non-Hodgkin's lymphoma, Hodgkin's lymphoma, acute myeloid leukemia (AML), B-lymphoid leukemia, blastic plasmocytoid dendritic neoplasm (BPDCN), or hairy cell leukemia.

In all embodiments of the methods and uses provided herein, the protein may comprise a heavy chain and a light chain, wherein the heavy chain comprises the amino acid sequence of SEQ ID NO: 25; and wherein the light chain comprises the amino acid sequence of SEQ ID NO: 26.

As used herein, the term “effective amount” or “therapeutically effective amount” refers to the amount of a pharmaceutical agent, e.g., a protein, a multimeric protein, an immunoconjugate, or a pharmaceutical composition disclosed herein, which is sufficient to reduce or ameliorate the severity and/or duration of a cancer, or one or more symptoms thereof, prevent the advancement of a disease, cause regression of a disease, prevent the recurrence, development, onset or progression of one or more symptoms associated with a disease, or enhance or improve the prophylactic or therapeutic effect(s) of another related therapy (e.g., prophylactic or therapeutic agent) for a cancer.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g., decisions on dosage etc., is within the responsibility of general practitioners and other medical doctors and may depend on the severity of the symptoms and/or progression of a disease being treated. Appropriate doses of antibody-based protein molecules are well known in the art (Ledermann J. A. et al., 1991, Int. J Cancer 47: 659-664; Bagshawe K. D. et al., 1991, Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody-based protein molecule may be determined by comparing its in vitro activity and in vivo activity in an animal model. Methods for extrapolation of effective dosages in mice and other test animals to humans are known. The precise dose will depend upon a number of factors, including whether the antibody-based protein is for prevention or for treatment, the size and location of the area to be treated, the precise nature of the antibody-based protein, and the nature of any detectable label or other molecule attached to the antibody-based protein.

A typical protein dose will be in the range 100 μg to 1 g for systemic applications, and 1 μg to 1 mg for intradermal injection. An initial higher loading dose, followed by one or more lower doses, may be administered. In some embodiments, the protein is an IgG1 or IgG4 isotype. A dose for a single treatment of an adult subject may be proportionally adjusted for children and infants. Treatments may be repeated at daily, twice-weekly, weekly or monthly intervals, at the discretion of the physician. The treatment schedule for a subject may be dependent on the pharmacokinetic and pharmacodynamic properties of the protein composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, a protein, a multimeric protein, an immunoconjugate, or a pharmaceutical composition disclosed herein may be administered as a sub-cutaneous injection. Sub-cutaneous injections may be administered using an auto-injector, for example for long term prophylaxis/treatment.

In some embodiments, the therapeutic effect of a protein, a multimeric protein, an immunoconjugate, or a pharmaceutical composition disclosed herein may persist for several half-lives, depending on the dose. For example, the therapeutic effect of a single dose of a protein, a multimeric protein, an immunoconjugate, or a pharmaceutical composition disclosed herein may persist in a subject for 1 month or more, 2 months or more, 3 months or more, 4 months or more, 5 months or more, or 6 months or more.

In some embodiments, a subject may be treated with a protein, a multimeric protein, an immunoconjugate, or a pharmaceutical composition disclosed herein and an additional therapeutic agent or therapy that is used to treat a cancer or a symptom or complication of a cancer. The protein, multimeric protein, immunoconjugate, or pharmaceutical composition disclosed herein and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Provided herein is a protein, a multimeric protein, an immunoconjugate, or a pharmaceutical composition disclosed herein, for use as a medicament.

Further provided herein is a protein, a multimeric protein, an immunoconjugate, or a pharmaceutical composition disclosed herein, for use in the treatment of a disease or a disorder.

Provided herein is a protein, a multimeric protein, an immunoconjugate, or a pharmaceutical composition disclosed herein for use in enhancing an anti-cancer immune response in a subject.

Provided herein is a protein, a multimeric protein, an immunoconjugate, or a pharmaceutical composition disclosed herein for use in treating cancer in a subject.

Provided herein is a protein, a multimeric protein, an immunoconjugate, or a pharmaceutical composition disclosed herein for use in ameliorating a symptom of cancer in a subject.

Provided herein is a protein, a multimeric protein, an immunoconjugate, or a pharmaceutical composition disclosed herein for use in ameliorating a symptom of cancer in a subject, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

Provided herein is a protein, a multimeric protein, an immunoconjugate, or a pharmaceutical composition disclosed herein for use in reducing size of a tumor in a subject.

Provided herein is a protein, a multimeric protein, an immunoconjugate, or a pharmaceutical composition disclosed herein for use in inhibiting the growth of a tumor in a subject.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% f a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular Systems Biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

As used herein, the term “conservative substitution” refers to replacement of an amino acid with another amino acid which does not significantly deleteriously change the functional activity. A preferred example of a “conservative substitution” is the replacement of one amino acid with another amino acid which has a value≥0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

The term “immunoconjugate” refer to a protein of the disclosure that is conjugated to a cytotoxic, a cytostatic and/or a therapeutic agent.

The term “isolated molecule” (where the molecule is, for example, a protein, a nucleic acid, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The terms “inhibit”, “block”, or “neutralize”, as used herein with respect to bioactivity of a protein disclosed herein means the ability of the protein to substantially antagonize, prohibit, restrain, slow, disrupt, eliminate, stop, reduce or reverse for example progression, strength, or severity of that which is being inhibited including, but not limited to, the binding of PD-L1 to PD-1, or the binding of CD47 to SIRPα.

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

PD-L1 is also known as programmed cell death ligand 1, CD274, B7-H, B7H1, PDCD1L1, PDCD1LG1, PDL1, and hPD-L1.

CD47 is also known as integrin-associated protein, IAP, MER6, and Antigenic Surface Determinant Protein OA3.

As used herein and unless otherwise stated, the terms “hinge linker”, “linker”, “hinge”, “first linker” and “second linker”, and derivations thereof, refer to a sequence, for example derived from an immunoglobulin hinge region, that can link two polypeptides, for example polypeptides of different Fab regions, and is separate from any hinge sequence in an immunoglobulin hinge region that may be part of a protein of the present invention.

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

As used herein, a “therapeutically effective amount” is the amount of a protein or a pharmaceutical composition provided herein that is effective to treat a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

The section headings used herein are for organizational purposes only and are not to be construed as limiting the subject matter described.

The disclosure will be further clarified by the following example, which is intended to be purely exemplary of the disclosure and in no way limiting.

### Example

### Generation of Optimized Conditionally Active Therapeutic Antibodies

### Introduction

In this example, we successfully generated and proved the utility of an optimized conditionally active antibody (LB101; sequences provided in Table 1). This conditionally active antibody was well expressed, biophysically stable, highly soluble and exhibited improved biological potency compared to an anti-PD-L1 IgG format antibody.

### Materials and Methods

### Cloning, Transient Expression, Purification and Characterization of Proteins

Antibody-encoding DNA sequences were cloned via restriction-ligation cloning into separate human IgG1 heavy and light-chain constant region-encoding expression cassettes in separate plasmid vectors, to create activatable constructs for expression. Produced antibodies were captured from clarified supernatants using a HiTrap MabSelect Sure Protein A 5 mL column on an ÄKTA Pure 150 L FPLC system. Eluted protein peaks were immediately buffer exchanged into 1× PBS pH 7.4 by directly loading the eluted protein A peak fractions onto a HiPrep 26/10 Desalting column. Protein concentration was determined by measuring the absorbance at 280 nm and 1 μg of each purified protein was analyzed by SDS-PAGE under reducing and/or non-reducing conditions using 4-20% TGX polyacrylamide gradient gels (BioRad, Cat. nr. 456-1093) with 1× Tris/glycine/SDS buffer, separated by 120V field for 1 hour. In order to test for the presence of non-covalently bound aggregates and to complement the SDS PAGE analysis, analytical size-exclusion chromatography was performed. Aliquots of selected clones were analyzed by analytical Size Exclusion Chromatography (SEC) using a Superdex 200 Increase 10/300 SEC column and 1× PBS pH 7.4 as running buffer, in isocratic mode. Selected proteins were further purified using preparative SEC. Antibody samples up to 1 ml were loaded onto a Superdex 200 Increase 10/300 SEC column or a HiLoad 26/600 Superdex 200 μg column equilibrated in 1× PBS pH 7.4. 1 ml fractions of peak of interest were collected, and main peak fractions were pooled. After size exclusion chromatography, the samples were again analyzed by SDS-PAGE, as above.

### Metalloprotease Digestion

Protein constructs were incubated for 0 to 24 hours at 37° C. with human Matrix Metalloprotease (MMP) enzyme MMP12 at a ratio of 1% total MMP to protein construct (wt/wt) in Tris buffered saline (pH7.4) containing 5 mM CaCl₂). The reactions were stopped by the addition of 20 mM EDTA and then samples tested for binding or functional activity as described.

### IgG Titration Binding ELISAs

To coat ELISA plates, target proteins were diluted to 1 μg/ml in PBS pH7.4 and added at 100 μl per well, at 4° C., o/n. Coated plates were washed 3× with PBS pH7.4, blocked with 4% Skim Milk Protein in PBS (380 μl/well) for 1 hr at room temperature (RT), then washed 5× with PBS-Tween 20 (PBST). Antibodies (100 μl/well; diluted in PBST) were then added and then incubated 1 hr at RT. Plates were then washed 3× with PBS and goat anti-human IgG-HRP added (100 μl/well) at RT, for 1 hr. Plates were then washed 3× with PBST and twice with PBS before the addition of 100 μl TMB per well. Reactions were stopped by adding 100 μl 2M H2S04/well and OD was read on a plate reader at 450 nm.

### Phagocytosis Potency Assays

Peripheral blood mononuclear cells (PBMCs) were isolated from whole blood by density gradient centrifugation. CD14 positive PBMCs were subsequently isolated via magnetic cell isolation using CD14 microbeads. In parallel, cancer cells were labelled using a green CFSE (carboxy-fluorescein diacetate, succinimidyl ester) cell tracer dye. A total of 6.25×10⁵ labelled Jurkat cells were pre-incubated in the presence of UH2 antibodies in 96 well plates for 1 hour at 37° C. in a humidified atmosphere containing 5% CO₂. Following incubation, 2.5×10⁵ CD14 positive cells were added to each well and incubated for a further hour under the same culture conditions. Cells were harvested by vigorous pipetting, stained with viability dye, fixed using ice-cold 4% paraformaldehyde for 10 minutes. Following fixation, cells were blocked with an Fc receptor binding inhibitor monoclonal antibody for 10 minutes and then incubated with an Alexa Fluor 647 (AF647) conjugated anti-human CD11b antibody at room temperature for 30 minutes and fixed a further time in 4% paraformaldehyde for 5 minutes. Cells were analysed on the BD Fortessa flow cytometer recording side scatter and forward scatter properties along with CFSE and AF647 fluorescence intensity data. Briefly, cell debris was gated out by scatter properties (SSC-Areaby FSC-Area). Single cells were also gated for by SSC-Area by SSC-Height and then by FSC-Area by FSC-Height. From the remaining single cell population, CFSE and CD11b double positives cells were gated using a quadrant gate placed based on the population of CD11b positive cells in the vehicle treated test. The percentage of CFSE positive cells from the CD11b positive population was calculated and plotted.

In Vivo Efficacy Analyses in hPD-L1+MC38 Syngeneic Tumor-Bearing C57B6/hPD-L1/hPD1 Mice

In in vivo multi-dose efficacy studies, IgG1 Isotype, Atezolizumab and LB101 were each dosed six times (on days 0, 3, 6, 9, 12, 15) in C57B6 mice transgenic for human PD-L1 and PD1, bearing tumors generated by sub-cutaneous inoculation with the mouse cancer cell line MC38 (stably transfected with human PD-L1). Dosing began once tumors were established at >100 mm². Tumor volumes were measured by caliper measurements. A repeat of this study was then performed, as above, with a full dose titration of LB101 (0.3, 1.5, 4.5 and 8.5 mg/kg).

### PD1/PD-L1 Cell-Based Antagonism Assay

The PD1/PD-L1 blockade cell-based bioassay (Promega), was used to measure the potency of antibodies in blocking the PD1/PD-L1 interaction. On the day before the assay, PD-L1 aAPC/CHO-K1 cells were thawed and transferred into cell recovery medium (90% Ham's F12/10% FBS). The cell suspension was dispensed to each of the inner 60 wells of two 96-well, white, flat-bottom assay plates, at 100 μl per well. Cell recovery medium was added to each of the outside wells and the assay plates and incubated overnight at 37° C./5% CO₂. On the day of the assay the sample IgGs were diluted 4-fold in assay buffer (99% RPMI 1640/1% FBS) from 100 nM to 0.01 nM and 40 μl per dilution added to the assay plates containing the PD-L1 aAPC/CHO-K1 cells. Positive inhibition control was theh Atezolizumab anti-PD-L1 antibody. As a negative inhibition control, an IgG1 isotype was included. PD1 Effector Cells were then thawed in assay buffer (99% RPMI 1640/1% FBS) and the cell suspension added to the wells of the assay plates containing the PD-L1 aAPC/CHO-K1 cells and the IgG titration samples. The assay plates were incubated for six hours in a 37° C./5% CO₂ incubator, allowed to equilibrate to ambient temperature for 5-10 minutes, then 80 μl of Bio-Glo™ Reagent (Promega) was added. Assay plates were incubated at ambient temperature for a further 5-30 minutes and luminescence signals subsequently measured at 10, 20 and 30 minutes.

### Assessment of Binding of LB101 to Human Fc Gamma Receptors

Binding of test antibodies to high and low affinity human Fc gamma receptors was assessed by single cycle analysis using a Biacore® T200, running at a flow rate of 30 μl/min. The human Fc receptors, FcγRI, FcγRIIA (both 167R and 167H polymorphisms), FcγRIIB, FcγRIIIA (both 176F and 176V polymorphisms) and FcγRIIIB were captured on a CM5 sensor chip pre-coupled using a His capture kit using standard amine chemistry. A five point, three-fold dilution range of test antibody (analyte) without regeneration between each concentration was used for each receptor tested. In all cases, test antibodies were passed over the chip in increasing concentrations at 30 l/min followed by a single dissociation step. Following dissociation, the chip was regenerated with injection of Glycine pH 1.5. The signal from the reference channel Fcl (blank) was subtracted from that of the Fc loaded with receptor to correct for differences in non-specific binding to the reference surface. Sensorgrams were analysed for 1:1 kinetics for the high affinity Fc gamma receptor hFcγRI and by steady state binding for the low affinity Fc gamma receptors.

### Affinity Analyses for Binding of LB101 to Human and Mouse CD47

In order to assess the binding of LB101, LB101 after 16 hr MMP12 digest, and positive control anti-CD47 Fab (recombinant Fab using the variable domain sequences used in LB101), to human and mouse CD47, multi-cycle kinetic analysis was performed at 25° C. on a Biacore® T200. HBS-P+ supplemented with 0.1% BSA was used as running buffer as well as for ligand and analyte dilutions. The LB101 antibodies and the control anti-CD47 Fab samples were diluted to 1.0 μg/mL in running buffer and at the start of each cycle loaded onto Fc2, Fc3 and Fc4 of a series S CM5 chip previously coupled with an anti-human Fab capture antibody using standard amine chemistry. Ligand was captured at a flow rate of 10 μl/min to give an immobilisation level (RL) of ˜ 60 RU. The surface was then allowed to stabilise. Multi-cycle kinetic data was obtained using either human or mouse CD47 as the analyte injected at a flow rate of 30 μl/min. An eight point, two-fold dilution range from 50.0 nM to 0.39 nM was prepared in running buffer for human CD47 and 500.0 nM to 3.9 nM for mouse CD47. For each concentration, the association phases were monitored for 180 seconds, and the dissociation phase was measured for 300 seconds. Regeneration of the sensor chip surface was conducted between cycles using 10 mM glycine pH 2.1. The signal from the reference channel Fcl (no ligand captured) was subtracted from that of Fc2-4 to correct for bulk effect and differences in non-specific binding to a reference surface. The signal from each CD47 blank run (ligand captured but no antigen) was subtracted to correct for differences in surface stability. Human CD47 binding was analysed using 1:1 binding analysis due to the high affinity interaction between the antibody and antigen. Mouse CD47 binding was analysed using steady state analysis.

### Results and Discussion

### Protein Construct Design Principles

A key issue that restricts efficacy in CD47-targeting drugs is that this protein is expressed on many different cell classes in the body, such as erythrocytes, thrombocytes and endothelial cells (amongst others). This off-tumor target expression often leads to dose-limiting side effect risks and antigen “sink” effects. The CD47 “sink” minimizes the amount of drug penetrating the tumor (even when large doses of IgG are given). The binding of blood cells by anti-CD47 is also a significant toxicity risk, potentially causing multiple forms of cytopenia and even hemagglutination in patients. These factors minimize the potential safety and efficacy of anti-CD47 antibodies.

The LB101 anti-PD-L1/CD47 protein constructs (FIG. 1) aim to overcome the peripheral sink and toxicity issues by minimizing binding of CD47 outside of diseased tissue. This effect is achieved by adding PD-L1 and linkers above (n-terminal to) the CD47 binding domains. The use of appropriate upper domain/linker combinations results in a configuration that fully blocks binding activity in the lower CD47 domain. The PD-L1 domain then drives high concentration in PD-L1 enriched tumor microenvironments, and the protein construct linker system exploits the elevated MMP and Cathepsin activity that is common in solid tumors to cleave the linker peptides, exposing the CD47 binding domains and thereby conditionally activating the CD47-binding activity in the tumor, rather than the periphery. The combined biological functions of LB101 thereby afford the molecule the potential to augment both innate and adaptive immune responses to cancer cells, as outlined in FIG. 2.

The LB101 design may be based on sequences derived from IgG1, IgG2, IgG3, IgG4, IgE, IgM, or IgA and may or may not have effector function capacity. In this construct, four polypeptide chains encode for four Fab domains (2× Fab A, 2× Fab B), four linker sequences, and may or may not have an immunoglobulin hinge region and an Fc domain. The linkers in the LB101 design are both proteolytically cleavable and may be sequentially cleaved, with a first “fast” cleavage taking the intact structure and creating an intermediate active state which allows Fabs A and B from a single protein construct to bind their cognate targets. Cleaved linkers derived from immunoglobulin hinge sequences may also recruit increased immune effector function (ADCC, CDC and ADCP) at the cell membrane via endogenous anti-hinge antibodies, which are a known phenomenon in human patients with (and even without) underlying autoreactive disease.

### Protein Construct Cloning, Expression, and Characterization

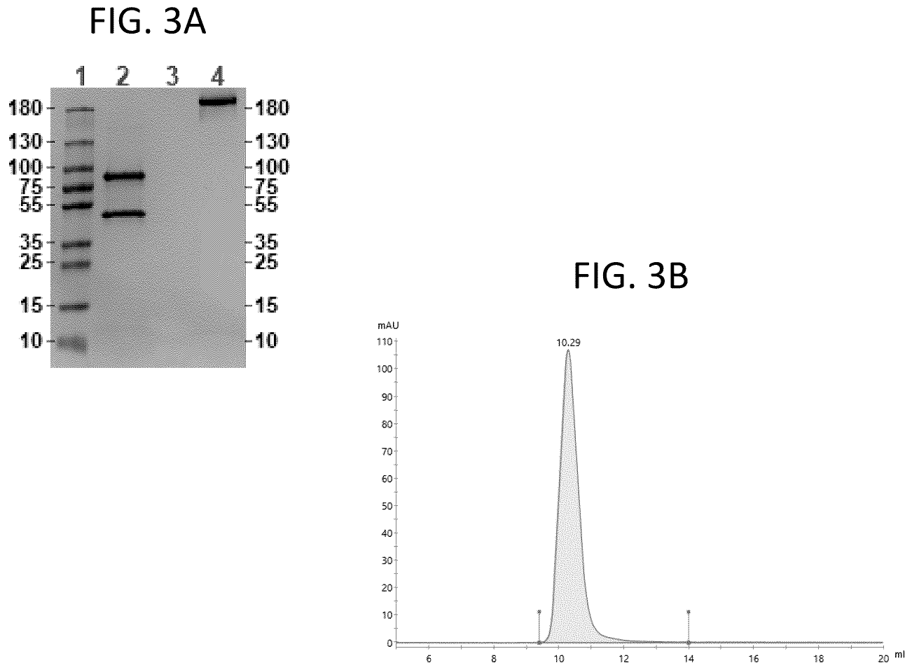

To produce LB101, DNA cassettes for each construct type (Table 1) were synthesized and cloned into expression vectors encoding human IgG1 heavy and light chain. The anti-PD-L1 variable domain sequences used in the protein construct disclosed herein are the variable domain sequences provided in U.S. Pat. No. 7,943,743 B2. The anti-CD47 variable domain sequences used in the protein construct disclosed herein are provided in U.S. Pat. No. 10,683,350 B2. The protein was produced by transient transfection of CHO cells, then purified by protein A affinity and size exclusion chromatographies. The purified protein demonstrated high purity and uniformity by SDS-PAGE (FIG. 3A) and analytical size-exclusion chromatography (SEC) (FIG. 3B), demonstrating that the LB101 construct can be expressed and purified as an intact, stable product, in a single process.

### LB101 In Vitro Functional Characterization

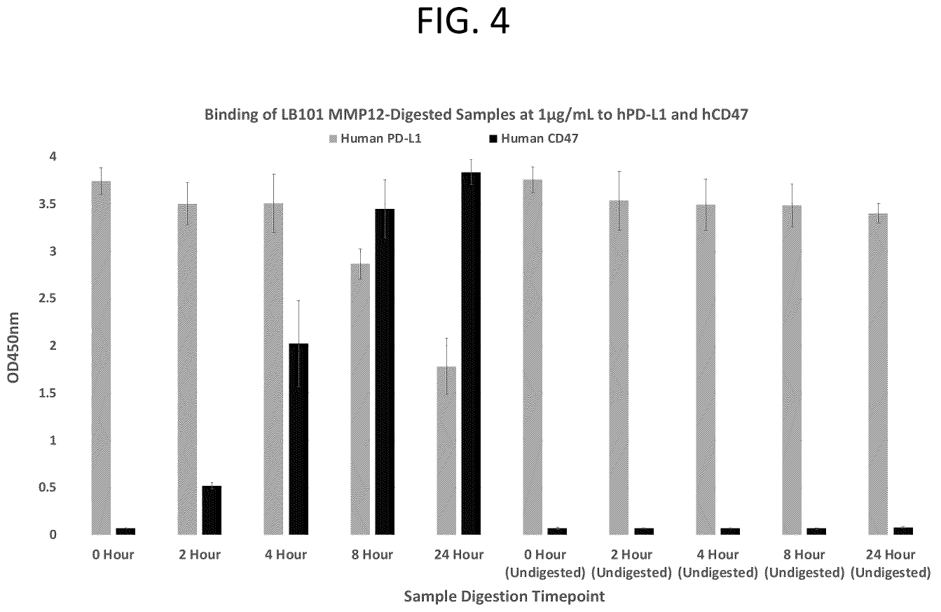

Purified LB101 protein was incubated at 37° C. for 0, 2, 4, 8 or 24 h in the presence or absence of human MMP12 enzyme, then tested for its binding signal on human PD-L1 and human CD47 by ELISA (FIG. 4). All samples exhibited high binding signal on human PD-L1, demonstrating that the LB101 domains which bind PD-L1 are fully active. Samples where MMP12 was not added did not exhibit any binding signal for CD47. Incubation with MMP12 for 2, 4, 8 and 24 hours demonstrated increased CD47 binding, suggesting this binding signal is induced by the cleavage of the linkers between the PD-L1 and CD47 domains.

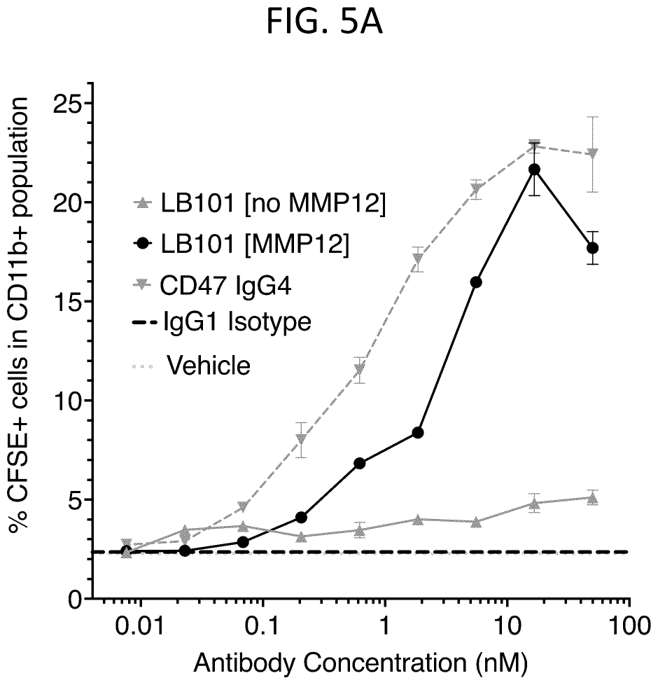

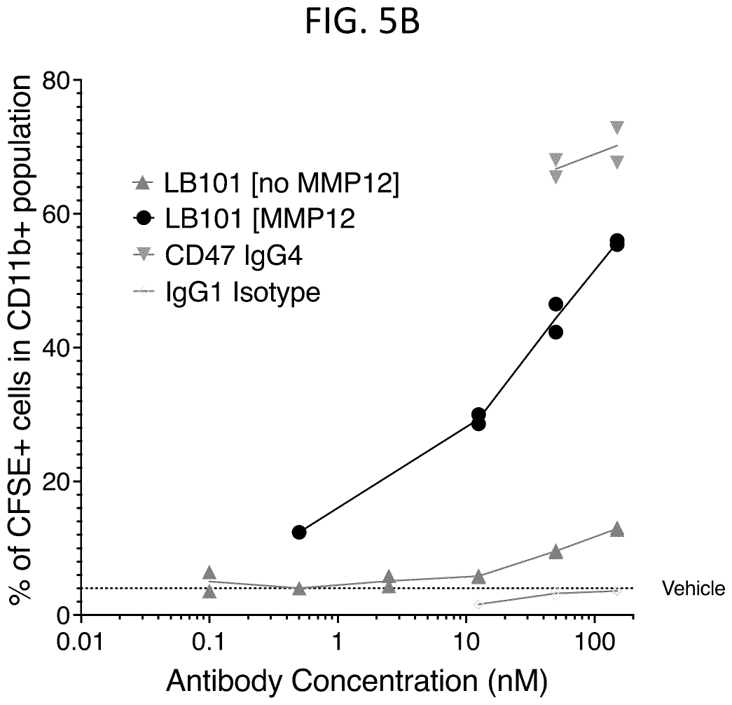

To examine the influence of CD47 binding and blocking by MMP12-activated LB101, CFSE-labelled A549 cells (which express PD-L1 and CD47) were mixed with freshly isolated human CD14+ cells in the presence of titrated IgG1 isotype, CD47 IgG4, LB101 without MMP12 treatment, LB101 with MMP12 treatment for 4 hours, and vehicle. Myeloid cells undergoing phagocytosis were observed by CD11b/CFSE staining in flow cytometry (FIG. 5A). This analysis showed all samples caused weak or no phagocytosis other than CD47 IgG4 positive control and LB101 with MMP12 treatment, demonstrating that the pro-phagocytic ability of LB101 is reliant on enzymatic activation. This phagocytosis assay was then repeated, using the PD-L1+/CD47+ myeloid cell line HEL as the target cell. Again, all samples caused weak or no phagocytosis other than CD47 IgG4 positive control and LB101 with MMP12 treatment, further demonstrating that the pro-phagocytic ability of LB101 is reliant on enzymatic activation and that the PD-L1 binding activity of LB101 is insufficient to drive strong phagocytic activity, alone.

### In Vivo Efficacy Analysis in MC38 Syngeneic Tumor Setting

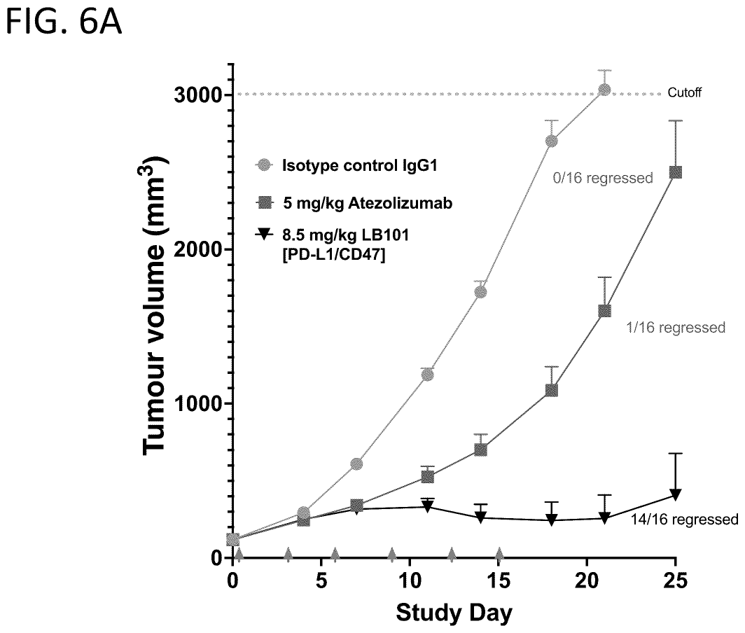

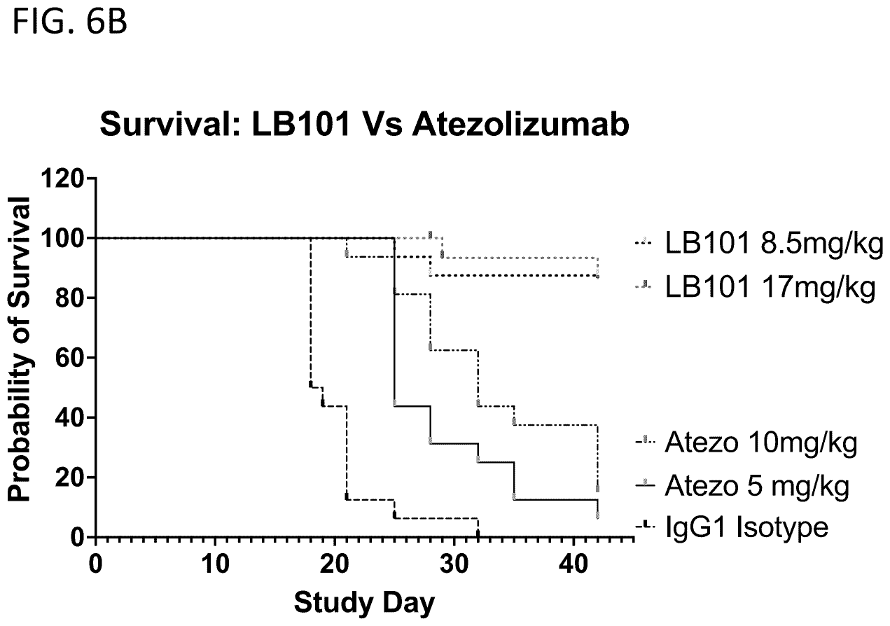

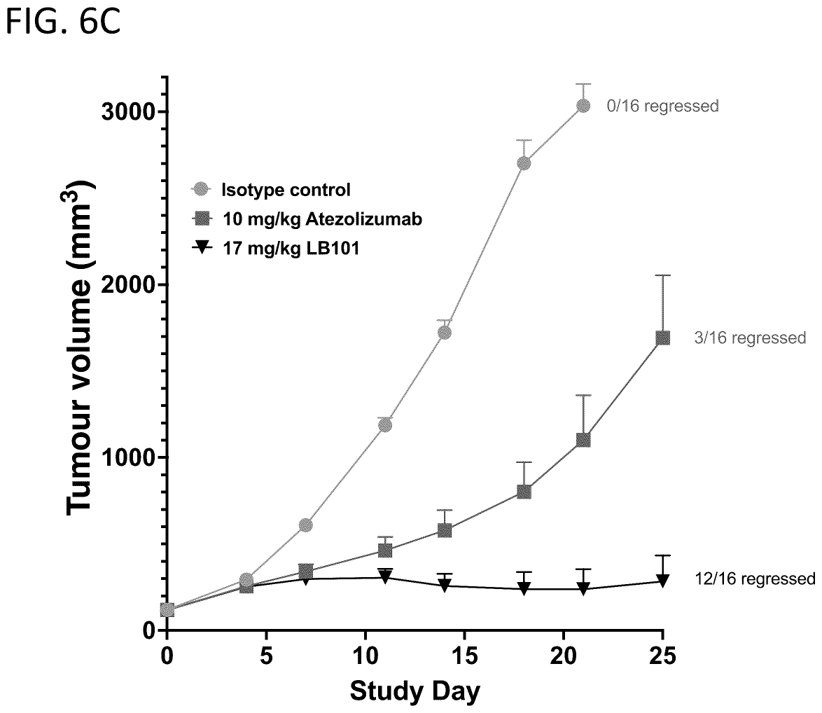

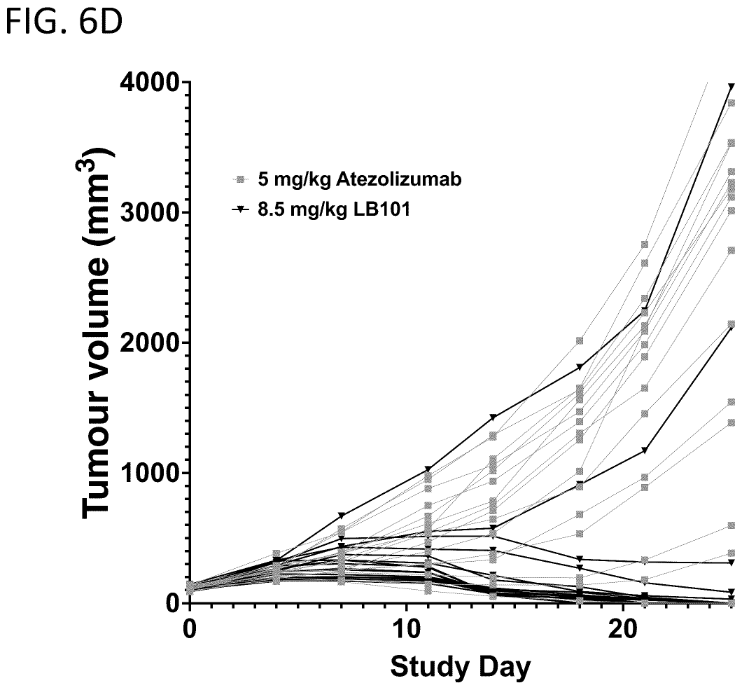

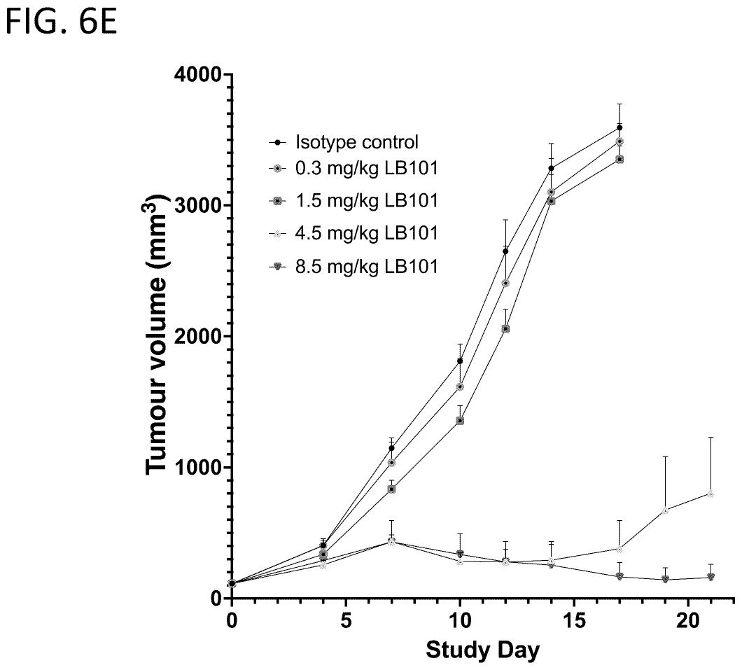

In in vivo multi-dose efficacy studies, equimolar doses of Isotype control IgG1 (5 mg/kg), LB101 (8.5 mg/kg) and Atezolizumab (5 mg/kg) were each dosed six times (every three days) in C57B6 mice transgenic for human PD-L1 and PD1, bearing tumors generated by sub-cutaneous inoculation with the mouse cancer cell line MC38 (stably transfected with human PD-L1). Dosing began once tumors were established at >100 mm² and animals were removed from the study at a cut off of 3000 mm². Tumor volumes were measured by caliper measurements (FIG. 6A). This study demonstrated that IgG1 isotype control allowed rapid and uniform outgrowth of the MC38 tumors, with 0/16 animals in this group exhibiting tumor regression. For the Atezolizumab group, tumor growth was slowed across the group, but only 1/16 in the group exhibited tumor regression. In the LB101 group, by contrast, tumor growth was strongly inhibited across the group, with 14/16 exhibiting tumor regression. This potency difference was also replicated in higher doses of Atezolizumab (10 mg/kg) and LB101 (17 mg/kg), with survival rates being similar to those of the 5 and 8.5 mg/kg groups outlined above (FIG. 6B, FIG. 6C), indicating that even high doses of Atezolizumab are incapable of achieving the potencies observed for LB101. The high potency of LB101 at a moderate dose of 8.5 mg/kg is therefore most likely driven by protease activation in the MC38 tumor microenvironment, leading to both PD-L1 and CD47 blockade and both adaptive and innate immune engagement being stimulated, as outlined in FIG. 2. This interpretation was further supported by the analysis of the tumor growth curves for individual mice in the 5 and 8.5 mg/kg groups (FIG. 6D), in which it was observed that some tumors continued to regress for e.g. 10 days after day 15 (when dosing ended).

A repeat of this study was then performed, as above, but with a full dose titration of LB101. In the dose titration analysis of LB101 versus isotype control IgG, both the 0.3 and 1.5 mg/kg doses were found to be sub-efficacious, while strong tumor growth inhibition was observed at 4.5 and strongest activity at 8.5 mg/kg (FIG. 6E).

### PD-L1 Bioactivity of LB101

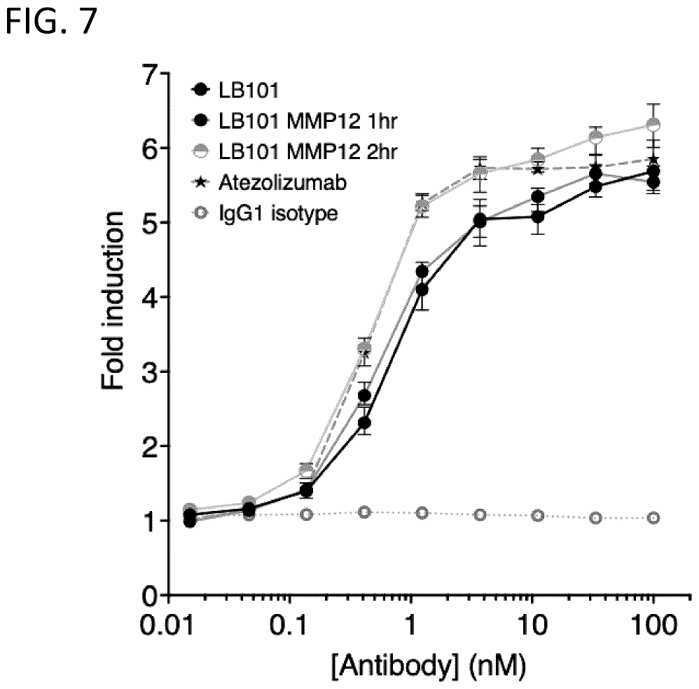

Isotype control, Atezolizumab and LB101 IgGs were tested for ability to antagonize PD-L1/PD1 signaling in the Promega PD1 cell signaling bioassay (FIG. 7). In this analysis, IgG1 isotype did not block PD-L1/PD1 binding, while Atezolizumab and LB101 (with or without MMP12 treatment for 1 or 2 hours) exhibited similarly potent signaling inhibition.

### “Rechallenge” Analysis in MC38 Syngeneic Tumor Setting

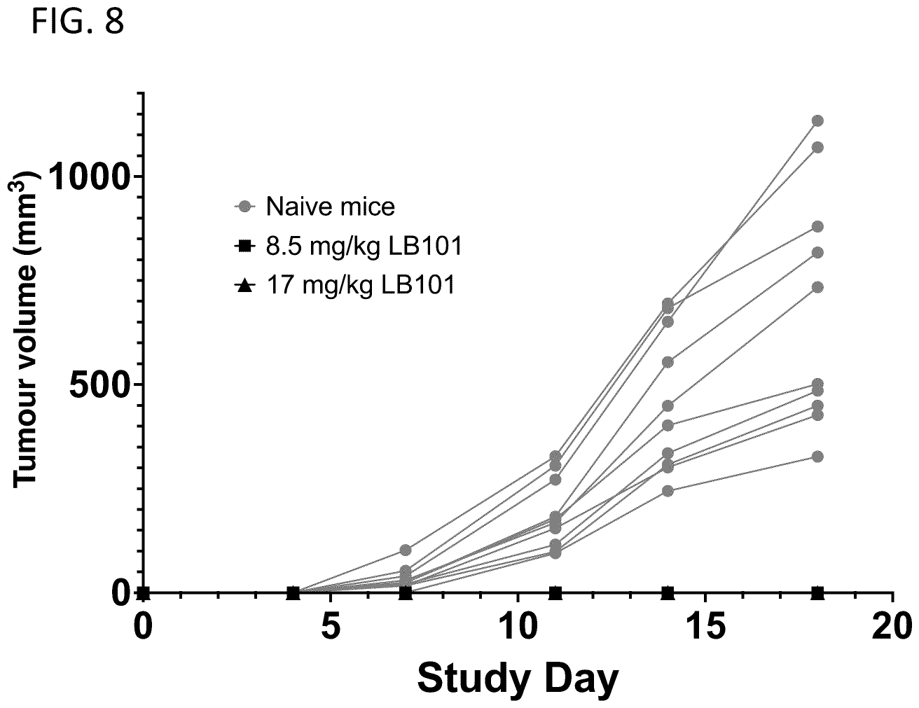

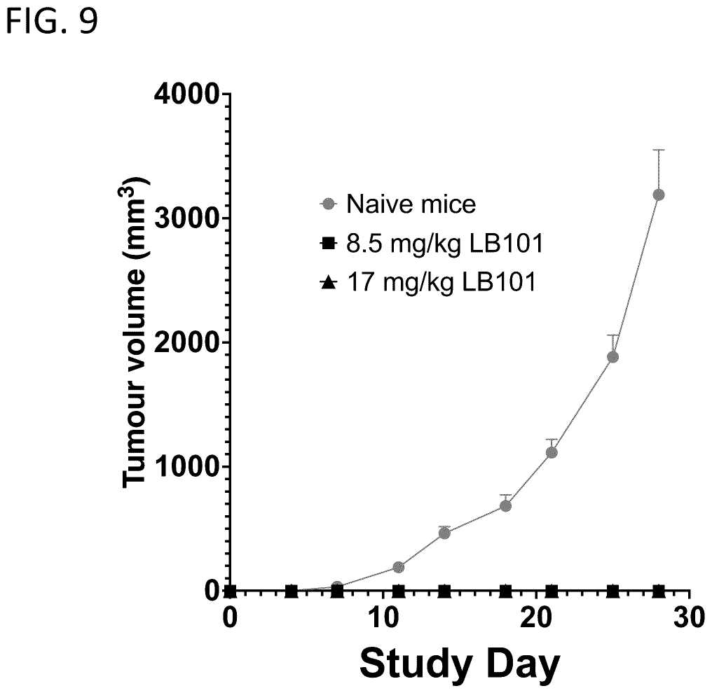

To perform a “rechallenge” experiment, naïve transgenic hPD-L1/hPD1 C57B6 mice (control group, n=10) and non-naïve mice that had previously exhibited complete hPD-L1+MC38 tumor regression (FIG. 6A, FIG. 6B) when treated with LB101 (8.5 mg/kg, n=13) or LB101 (17 mg/kg, n=9) were reimplanted with hPD-L1+MC38 cells and tumor growth measured over 18 days (FIG. 8). This analysis demonstrated that no LB101-treated animals grew tumors. In contrast, all the control animals (10/10) established tumors with sizes ranging from 330-1100 mm³ by day 18. By day 28, all the control animals (10/10) established tumors with mean size of approximately 3000 mm³, while all LB101 animals remained tumor free (FIG. 9).

### Analysis of LB101 Immunomodulatory Effects in MC38 Syngeneic Tumor Setting

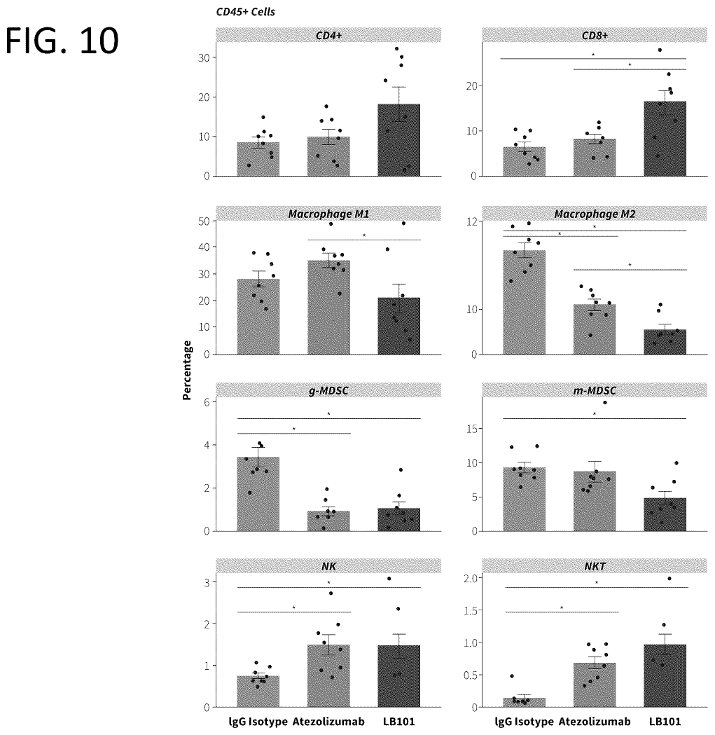

To examine the immunomodulatory effects of LB101 in the tumor in comparison to controls, equimolar doses of Isotype control IgG1 (5 mg/kg), LB101 (8.5 mg/kg) and Atezolizumab (5 mg/kg) were each dosed 4 times (every three days) in C57B6 mice transgenic for human PD-L1 and PD1 (n=8 mice per group), bearing tumors generated by subcutaneous inoculation with the mouse cancer cell line MC38 (stably transfected with human PD-L1). Dosing began once tumors were established at >100 mm². After 4 doses, tumor samples from all mice were extracted and immune cell infiltrate levels were measured by flow cytometry. LB101 induced a significant (p<0.05) increase in CD4+ and CD8+ T cells and natural killer (NK) cells, and reduction in immunosuppressive myeloid-derived suppressor cells and macrophages compared to IgG isotype in tumors (FIG. 10). Importantly, LB101 also induced a significant (p<0.05) increase in CD8+ T cells, and reduction in immunosuppressive m-MDSC and M2 macrophages compared to Atezolizumab in tumors, illustrating differentiation in the potency of immunomodulation mediated by LB101 in comparison to a standard anti-PD-L1 IgG (FIG. 10).

### Analysis of LB101 Pharmacokinetics and Safety in Cynomolgus Monkey

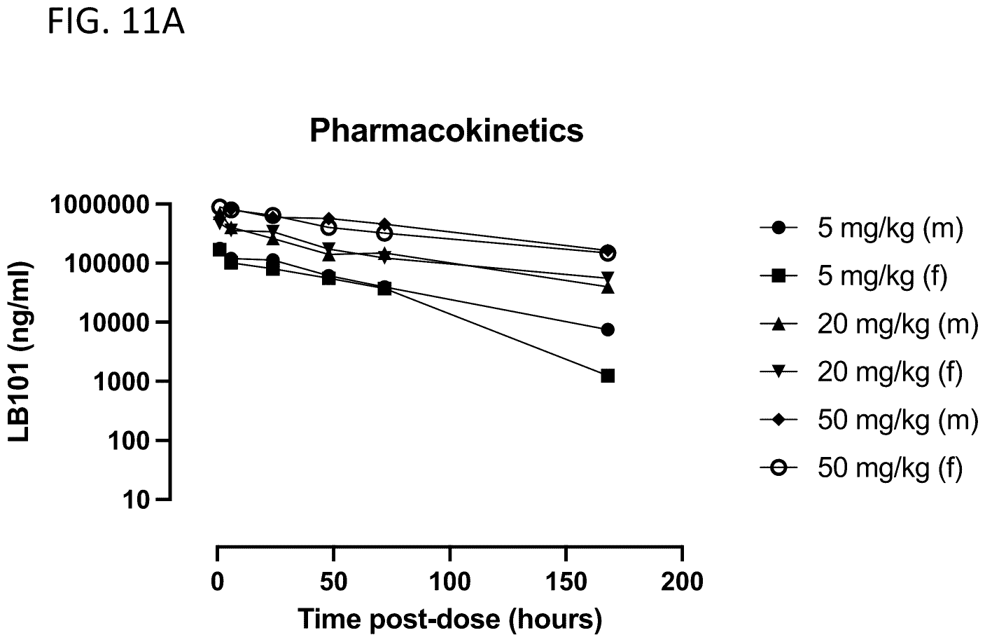

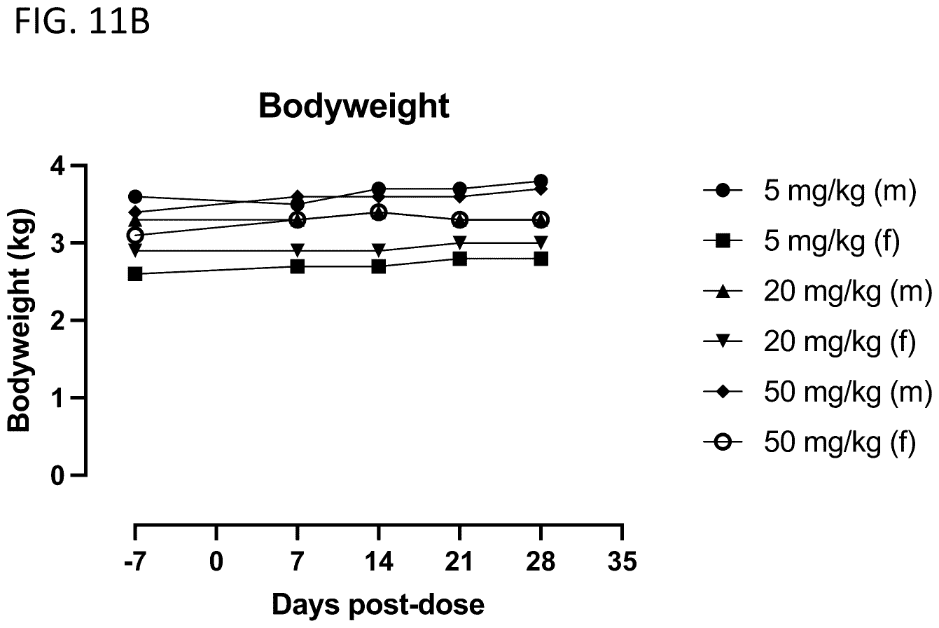

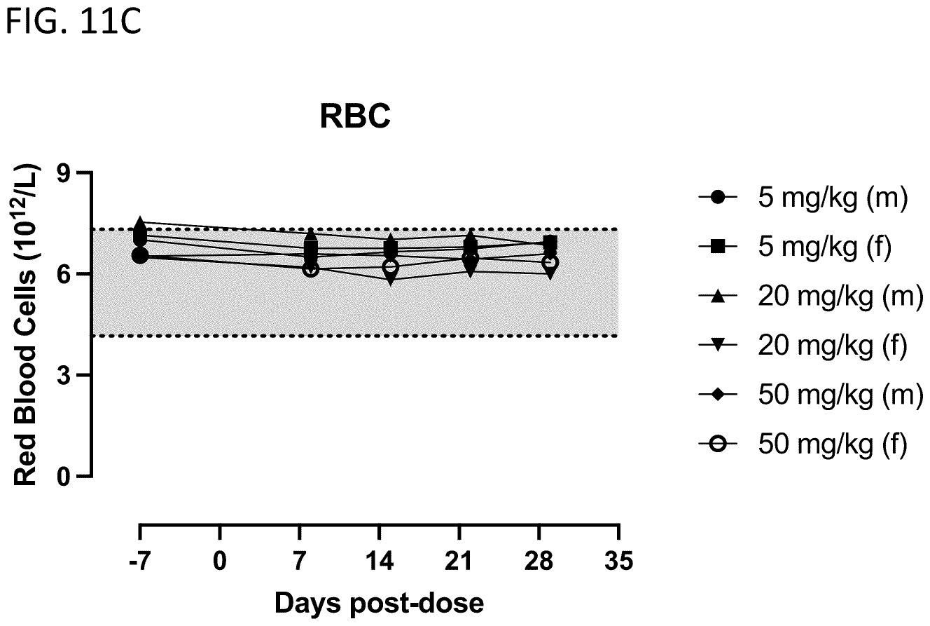

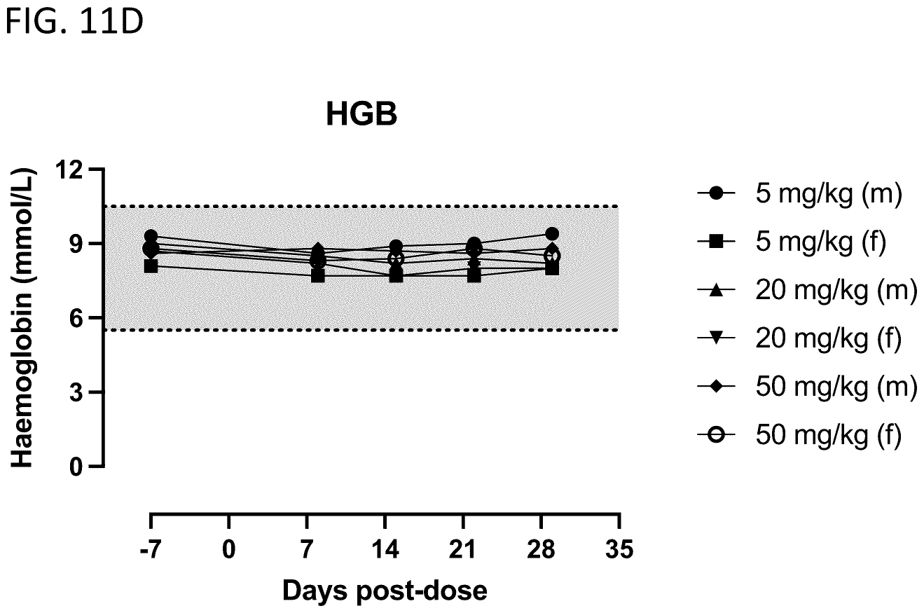

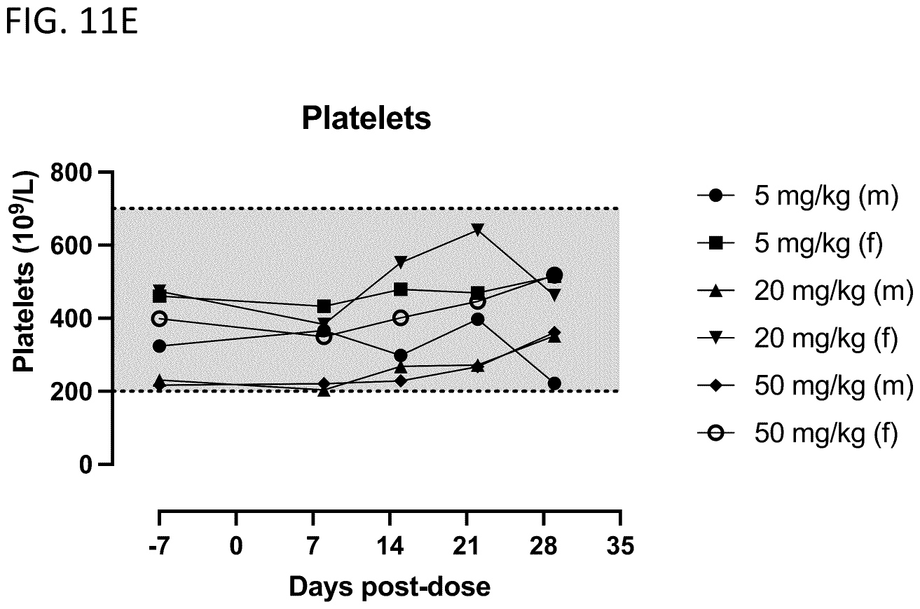

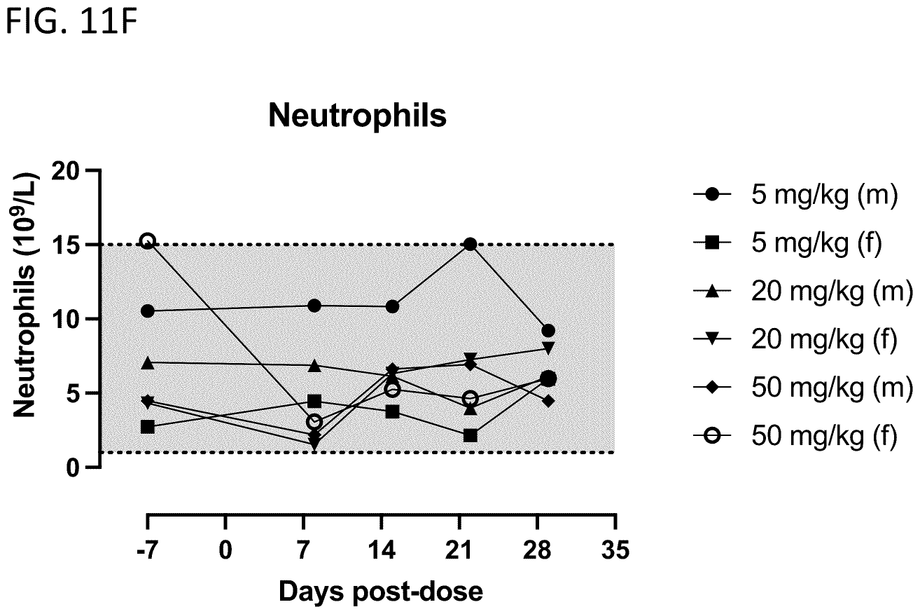

A dose range finding (DRF) study of LB101 in cynomolgus monkeys was performed in order to establish pharmacokinetics and safety parameters. Male and Female monkeys were dosed four times, once every seven days, at 5, 20 or 50 mg/kg of LB101. Blood samples were taken and measurements were made of LB101 pharmacokinetics (FIG. 11A), which demonstrated that LB101 exhibits linear, dose-dependent concentration over 7 days post-dose. Body weight measurements (FIG. 11B) did not exhibit any weight loss in any animal. Most importantly, factors known to be at risk of change due to CD47 blockade such as Red Blood Cell levels (RBC, FIG. 11C), Hemoglobin levels (HBG, FIG. 11D), Platelet levels (FIG. 11E) and Neutrophil levels (FIG. 11F) all remained unchanged and within normal levels, over the full dosing scheme. These findings demonstrated that the CD47 binding capacity of LB101 was indeed fully restricted in the periphery and activation levels of LB101 are extremely low, as CD47-blocking IgGs are associated with nonlinear PK, and severe anemia at doses above 1 mg/kg.

### Assessment of Binding of LB101 to Human Fc Gamma Receptors

Binding to high and low affinity human Fe gamma receptors of control antibodies, LB101, and LB101 digested with MMP12, was examined using Biacore® biosensor technology (Table 2). Significant binding of the positive control human IgG1 trastuzumab was observed to both the high and low affinity human Fc gamma receptors. LB101 was found to bind with similar affinity to Trastuzumab, for all receptors. Overall, LB101 digested for 2 h with MMP12 retained the ability to bind to all Fc gamma receptors with similar affinity to LB101, albeit that LB101 MMP12 digested exhibited slightly reduced affinity (KD>3-fold) to hFcγRIIA167 and hFcγRIIB (Table 2).

TABLE 2
Biacore ® - human Fcγ receptors

| Receptor | Antibody | KD (M) | Chi2 |
|---|---|---|---|
| FcγRI | Trastuzumab | 1.93E−09 | 1.66 |
| | LB101 | 2.59E−09 | 2.51 |
| | LB101 [MMP12 2 h] | 3.08E−09 | 0.879 |
| FcγRIIA | Trastuzumab | 1.27E−06 | 24.4 |
| (His167) | LB101 | 1.23E−06 | 0.837 |
| | LB101 [MMP12 2 h] | 4.14E−06 | 7.92 |
| FcγRIIA | Trastuzumab | 3.28E−06 | 11.6 |
| (Arg167) | LB101 | 3.86E−06 | 5.28 |
| | LB101 [MMP12 2 h] | 2.46E−06 | 11.3 |
| FcγRIIB | Trastuzumab | 9.00E−06 | 25.4 |
| | LB101 | 3.97E−06 | 0.19 |
| | LB101 [MMP12 2 h] | 1.48E−05 | 3.6 |
| FcγRIIIA | Trastuzumab | 7.26E−07 | 0.688 |
| (Phe176) | LB101 | 1.11E−06 | 6.82 |
| | LB101 [MMP12 2 h] | 8.76E−07 | 0.914 |
| FcγRIIIA | Trastuzumab | 1.72E−07 | 0.715 |
| (Val176) | LB101 | 2.33E−07 | 2.54 |
| | LB101 [MMP12 2 h] | 4.99E−07 | 9.4 |

### Affinity Analyses for Binding of LB101 to Human and Mouse CD47

Table 3 summarizes the data for LB101, LB101 after 16 hr MMP12 digest, and positive control anti-CD47 Fab, to human and mouse CD47. The LB101 sample demonstrated no binding to either human or mouse CD47. The unlocked LB101 (16 hr digest) bound to both human and mouse CD47. As the affinity for mouse CD47 was found to be lower that for human CD47, the steady state analysis method was used to derive the K_{D} value.

TABLE 3
Biacore ® - affinity for human and mouse CD47

| Receptor | Antibody | ka (1/Ms) | kd (1/s) | KD (M) | Chi2 |
|---|---|---|---|---|---|
| human | CD47 Fab | 1.57E−06 | 1.47E−04 | 9.37E−11 | 1.05 |
| CD47 | LB101 | NB | NB | NB | NB |
| | LB101 [MMP12 | 1.17E−06 | 4.09E−04 | 3.50E−10 | 0.515 |
| | 16 h] | | | | |
| mouse | CD47 Fab | NA | NA | 1.65E−07 | 1.17 |
| CD47 | LB101 | NB | NB | NB | NB |
| | LB101 [MMP12 | NA | NA | 2.27E−07 | 0.916 |
| | 16 h | | | | |

NB = No binding observed
NA = Not applicable (steady state binding method)

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention. The disclosures of each patent, patent application, and publication cited or described in this document are hereby incorporated herein by reference, each in its entirety, for all purposes.

### Numbered Embodiments

Notwithstanding the appended claims, the disclosure sets forth the following numbered embodiments:

Embodiment 1. A protein comprising a heavy chain and a light chain, wherein the heavy chain comprises, in N-terminus to C-terminus order, an anti-PD-L1 heavy chain variable (VH) domain, a CH domain, a first linker, an anti-CD47 VH domain, and an immunoglobulin heavy chain constant region;
wherein the light chain comprises, in N-terminus to C-terminus order, an anti-PD-L1 light chain variable (VL) domain, a first immunoglobulin light chain constant region, a second linker, an anti-CD47 VL domain, and a second immunoglobulin light chain constant region;
wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3;
wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6;
wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9;
wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12;
wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and
wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Embodiment 2. The protein of embodiment 1, wherein the anti-PD-L1 VH domain comprises the amino acid sequence of SEQ ID NO: 21, and the anti-PD-L1 VL domain comprises the amino acid sequence of SEQ ID NO: 22; and wherein the anti-CD47 VH domain comprises the amino acid sequence of SEQ ID NO: 23, and the anti-CD47 VL domain comprises the amino acid sequence of SEQ ID NO: 24.

Embodiment 3. The protein of embodiment 1 or 2, wherein the heavy chain comprises the amino acid sequence of SEQ ID NO: 25; and wherein the light chain comprises the amino acid sequence of SEQ ID NO: 26.

Embodiment 4. The protein of embodiment 1 or 2, wherein the immunoglobulin heavy chain constant region is an IgG, IgE, IgM, IgD, IgA, or IgY constant region.

Embodiment 5. The protein of embodiment 1 or 2, wherein the immunoglobulin heavy chain constant region is an IgG1, IgG2, IgG3, IgG4, IgA1 or IgA2 constant region.

Embodiment 6. The protein of embodiment 1 or 2, wherein the immunoglobulin heavy chain constant region is immunologically inert.

Embodiment 7. The protein of embodiment 1 or 2, wherein the immunoglobulin heavy chain constant region is a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.

Embodiment 8. The protein of any one of embodiments 1-7, wherein the first linker comprises the amino acid sequence of SEQ ID NO: 13.

Embodiment 9. The protein of any one of embodiments 1-8, wherein the second linker comprises the amino acid sequence of SEQ ID NO: 14.

Embodiment 10. A multimeric protein that is a dimer of two identical proteins, wherein each protein is the protein of any one of embodiments 1-9.

Embodiment 11. An immunoconjugate comprising the protein of any one of embodiments 1-9, linked to a therapeutic agent.

Embodiment 12. The immunoconjugate of embodiment 11, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Embodiment 13. A pharmaceutical composition comprising the protein of any one of embodiments 1-9, the multimeric protein of embodiment 10, or the immunoconjugate of embodiment 11 or 12, and a pharmaceutically acceptable carrier, diluent or excipient.

Embodiment 14. A nucleic acid molecule encoding (a) the heavy chain amino acid sequence; (b) the light chain amino acid sequence; or (c) both the heavy chain and the light chain amino acid sequences of the protein of any one of embodiments 1-9.

Embodiment 15. The nucleic acid molecule of embodiment 14, wherein the nucleic acid molecule comprises the nucleotide sequence of SEQ ID NO: 27, the nucleotide sequence SEQ ID NO: 28, or the nucleotide sequences of both SEQ ID NO: 27 and SEQ ID NO: 28.

Embodiment 16. An expression vector comprising the nucleic acid molecule of embodiment 15.

Embodiment 17. A recombinant host cell comprising the nucleic acid molecule of embodiment 15 or the expression vector of embodiment 16.

Embodiment 18. A method of producing a protein, the method comprising: culturing a recombinant host cell comprising the expression vector of embodiment 16 under conditions whereby the nucleic acid molecule is expressed, thereby producing the protein; and isolating the protein from the host cell or culture.

Embodiment 19. A method for enhancing an anti-cancer immune response in a subject, the method comprising administering to the subject a therapeutically effective amount of the protein of any one of embodiments 1-9, the multimeric protein of embodiment 10, the immunoconjugate of embodiment 11 or 12, or the pharmaceutical composition of embodiment 13.

Embodiment 20. A method for treating cancer in a subject, the method comprising administering to the subject a therapeutically effective amount of the protein of any one of embodiments 1-9, the multimeric protein of embodiment 10, the immunoconjugate of embodiment 11 or 12, or the pharmaceutical composition of embodiment 13.

Embodiment 21. A method for ameliorating a symptom of cancer in a subject, the method comprising administering to the subject a therapeutically effective amount of the protein of any one of embodiments 1-9, the multimeric protein of embodiment 10, the immunoconjugate of embodiment 11 or 12, or the pharmaceutical composition of embodiment 13.

Embodiment 22. The method of any one of embodiments 19-21, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

Embodiment 23. A method for reducing the size of a tumor in a subject, the method comprising administering to the subject a therapeutically effective amount of the protein of any one of embodiments 1-9, the multimeric protein of embodiment 10, the immunoconjugate of embodiment 11 or 12, or the pharmaceutical composition of embodiment 13.

Embodiment 24. A method for inhibiting the growth of a tumor in a subject, the method comprising administering to the subject a therapeutically effective amount of the protein of any one of embodiments 1-9, the multimeric protein of embodiment 10, the immunoconjugate of embodiment 11 or 12, or the pharmaceutical composition of embodiment 13.

Embodiment 25. A protein of any one of embodiments 1-9, a multimeric protein of embodiment 10, an immunoconjugate of embodiment 11 or 12, or a pharmaceutical composition of embodiment 13 for use in enhancing an anti-cancer immune response in a subject.

Embodiment 26. A protein of any one of embodiments 1-9, a multimeric protein of embodiment 10, an immunoconjugate of embodiment 11 or 12, or a pharmaceutical composition of embodiment 13 for use in treating cancer in a subject.

Embodiment 27. A protein of any one of embodiments 1-9, a multimeric protein of embodiment 10, an immunoconjugate of embodiment 11 or 12, or a pharmaceutical composition of embodiment 13 for use in ameliorating a symptom of cancer in a subject.

Embodiment 28. A protein, a multimeric protein, an immunoconjugate, or a pharmaceutical composition of any one of embodiments 25-26 for use wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

Embodiment 29. A protein of any one of embodiments 1-9, a multimeric protein of embodiment 10, an immunoconjugate of embodiment 11 or 12, or a pharmaceutical composition of embodiment 13 for use in reducing size of a tumor in a subject.

Embodiment 30. A protein of any one of embodiments 1-9, a multimeric protein of embodiment 10, an immunoconjugate of embodiment 11 or 12, or a pharmaceutical composition of embodiment 13 for use in inhibiting the growth of a tumor in a subject.

1. A protein comprising a heavy chain and a light chain,
wherein the heavy chain comprises, in N-terminus to C-terminus order, an anti-PD-L1 heavy chain variable (VH) domain, a CH domain, a first linker, an anti-CD47 VH domain, and an immunoglobulin heavy chain constant region;
wherein the light chain comprises, in N-terminus to C-terminus order, an anti-PD-L1 light chain variable (VL) domain, a first immunoglobulin light chain constant region, a second linker, an anti-CD47 VL domain, and a second immunoglobulin light chain constant region;
wherein the anti-PD-L1 VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3;
wherein the anti-PD-L1 VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6;
wherein the anti-CD47 VH domain comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9;
wherein the anti-CD47 VL domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12;
wherein the first linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20; and
wherein the second linker comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

2. The protein of claim 1,
wherein the anti-PD-L1 VH domain comprises the amino acid sequence of SEQ ID NO: 21, and the anti-PD-L1 VL domain comprises the amino acid sequence of SEQ ID NO: 22; and
wherein the anti-CD47 VH domain comprises the amino acid sequence of SEQ ID NO: 23, and the anti-CD47 VL domain comprises the amino acid sequence of SEQ ID NO: 24.

3. The protein of claim 1,
wherein the heavy chain comprises the amino acid sequence of SEQ ID NO: 25; and
wherein the light chain comprises the amino acid sequence of SEQ ID NO: 26.

4. The protein of claim 1, wherein the immunoglobulin heavy chain constant region is an IgG, IgE, IgM, IgD, IgA, or IgY constant region.

5. The protein of claim 1, wherein the immunoglobulin heavy chain constant region is an IgG1, IgG2, IgG3, IgG4, IgA1 or IgA2 constant region.

6. The protein of claim 1, wherein the immunoglobulin heavy chain constant region is immunologically inert.

7. The protein of claim 1, wherein the immunoglobulin heavy chain constant region is a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.

8. The protein of claim 1, wherein the first linker comprises the amino acid sequence of SEQ ID NO: 13.

9. The protein of claim 1, wherein the second linker comprises the amino acid sequence of SEQ ID NO: 14.

10.-12. (canceled)

13. A pharmaceutical composition comprising the protein of claim 1, and a pharmaceutically acceptable carrier, diluent or excipient.

14.-19. (canceled)

20. A method for treating cancer in a subject, the method comprising administering to the subject a therapeutically effective amount of the protein of claim 1.

21. (canceled)

22. The method of claim 20, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

23.-30. (canceled)

31. The protein of claim 1, wherein the the anti-PD-L1 VH domain comprises the amino acid sequence of SEQ ID NO: 21, and the anti-PD-L1 VL domain comprises the amino acid sequence of SEQ ID NO: 22.

32. The protein of claim 1, wherein the anti-CD47 VH domain comprises the amino acid sequence of SEQ ID NO: 23, and the anti-CD47 VL domain comprises the amino acid sequence of SEQ ID NO: 24.

33. The protein of claim 1, wherein the heavy chain comprises an amino acid sequence having at least 90% sequence identity to the amino acid sequence of SEQ ID NO: 25.

34. The protein of claim 1, wherein the light chain comprises an amino acid sequence having at least 90% sequence identity to the amino acid sequence of SEQ ID NO: 26.

35. The protein of claim 1, wherein the heavy chain comprises an amino acid sequence encoded by a nucleic acid having at least 90% sequence identity to the nucleic acid sequence of SEQ ID NO: 27.

36. The protein of claim 1, wherein the light chain comprises an amino acid sequence encoded by a nucleic acid having at least 90% sequence identity to the nucleic acid sequence of SEQ ID NO: 28.

37. The protein of claim 1, wherein the first linker or the second linker is a cleavable linker and wherein the cleavable linker is cleavable by a protease.

38. The protein of claim 37, wherein the protease is a MMP or a cathepsin.
